(19) United States
(12) Patent Application Publication
(10) Pub. No.: US 2016/0354411 A1
(43) Pub. Date: Dec. 8, 2016

(54) OLIVAMINE-INDUCED IMPROVEMENT IN ENDOTHELIAL CELLS VIABILITY AND FUNCTION

(71) Applicant: Darlene E. McCord, Coralville, IA (US)

(72) Inventor: Darlene E. McCord, Coralville, IA (US)

(21) Appl. No.: 14/731,441

(22) Filed: Jun. 5, 2015

(51) Int. Cl. A61K 35/36; A61K 31/401; A61K 31/198; A61K 31/05; A61K 31/7048

(52) U.S. Cl. CPC A61K 35/36; A61K 31/05; A61K 31/7048; A61K 31/198; A61K 31/401

(57) ABSTRACT

Compositions for and methods of improving wound healing are disclosed, including compositions for and methods of treating chronic wounds, and compositions for the inhibition and treatment of necrosis and extended quiescence that result in cellular necrosis instead of normal proliferation. The compositions include hydroxytyrosol, oleuropein, or a combination of hydroxytyrosol and oleuropein with endothelial cells. The invention further encompasses methods involving administration of these compositions.

### FIELD OF THE INVENTION

The present invention generally relates to compositions and methods for protecting and/or improving the health and function of endothelial cells and, in particular, to angiogenic capacity of vascular endothelial cells. The compositions and methods may be used, for example, in the treatment of skin that is distressed or wounded as result of a disease or other biological condition or process. Beneficially, the improved methods for wound healing are provided in combination with a regenerative therapy, namely use of endothelial cells in combination with hydroxytyrosol and/or oleuropein-containing formulations.

### BACKGROUND OF THE INVENTION

The epidermis is the outermost layer of the skin and forms the protective wrap over the body's surface. The epidermis can be further subdivided into strata with the outermost layer of the epidermis being the stratum corneum which is responsible for keeping water in the body and keeping harmful chemicals and pathogens out, making skin a natural barrier to infection. Transepidermal water loss, i.e., water that passes from inside a body (animal or plant) through the epidermal layer (skin) to the surrounding atmosphere via diffusion and evaporation processes, is a normal part of the cellular activity and regulated by the stratum corneum. Excessive transepidermal water loss, however, activates an inflammatory response in the epidermis and the dermis.

Corneotherapy is a skin care concept based on repairing the stratum corneum and therefore improving the function of the skin barrier. Topically applied substances influence the biochemistry in the horny layer of the skin and subsequent processes in deeper skin layers, which consequently have effects on the constitution of the horny layer, creating a cyclical effect that starts at the surface of the skin. A healthy and functioning skin barrier provides overall protection against dehydration and the penetration of germs, allergens, irritants, radicals, and radiation. This protection supports a gradual reduction in inflammation and other skin problems as the external causative agents are repelled by an intact skin barrier.

Wound healing, or wound repair, is a process in which the skin repairs itself after injury. In normal skin, the epidermis (outermost layer) and dermis (inner or deeper layer) exist in a steady-stated equilibrium, forming a protective barrier against the external environment. Once the protective barrier is broken, the normal (physiologic) process of wound healing is immediately set in motion.

The wound healing process is susceptible to interruption or failure leading to the formation of chronic non-healing wounds, that is, a wound that does not heal in an orderly manner and in a predictable amount of time as compared to wounds resulting from surgery (also sometimes known as wounds of primary intention) or wounds caused by trauma; for example, wounds that do not heal within several months are often considered chronic. Chronic wounds present a particularly difficult problem to treat and are typically classified into three categories: venous ulcers, diabetic, and pressure ulcers. A small number of wounds that do not fall into these categories may be due to causes such as radiation poisoning or ischemia. Chronic wounds, especially ulcerative wounds such as pressure ulcers (bed sores), diabetic ulcers, venous ulcers, etc. that, without treatment, are often trapped in the inflammation phase of wound healing. These types of wounds often accelerate quickly and damage not only the skin, but underlying tissues as well. The excessive healing time required for these types of wounds can lead to secondary complications, such as permanent underlying tissue damage, nerve damage, loss of circulation, and even mortality.

Pressure ulcers and certain other chronic wounds are sometimes categorized according to severity by the use of stages. According to one protocol, Stage I is characterized by a surface reddening of the skin; to the unaided eye, the skin is unbroken and the wound is superficial. Stage II is characterized by a partial thickness skin loss involving the dermis and/or epidermis, typically presenting as an abrasion, blister (broken or unbroken), shallow crater or other lesion, that is visible to the unaided eye. Stage III wounds extend through all of the layers of the skin and are a primary site for a serious infection to occur. Stage IV wounds extend through the skin and involves underlying muscle, tendons and bone. The term “peripheral to the wound” or “peri-wound area” refers to the area adjacent to a wound (a Stage II, III or IV wound) and typically extends from immediately adjacent the wound up to about 3 to 5 cm.

Oxidative stress is closely associated with mitochondrial dysfunction; however the detailed mechanism of this link requires further research. Olive phenols including oleuropein (OL) and hydroxytyrosol (HT) have been discovered to impart protective effects on vascular endothelial cells by attenuating oxidative injury and inflammatory damage mediated by TNF-α, thereby supporting vascular cell proliferation. In contrast, these phenols have also been shown to abrogate angiogenesis through the inhibition of matrix metalloproteinase-2 (MMP-2) and metallopeptidase 9 (MMP-9) activity, as well as the down regulation of vascular endothelial growth factor (VEGF) expression. However, these inhibitory effects on neovascularisation have generally been found in vitro using high concentrations of HT (≧100 μM). Abnormal vessel growth which promotes tumorigenesis has been partly attributed to aberrant VEGF expression, which differs from the processes within normal wounds, where conserved angiogenic mechanisms allow for adequate tissue repair and neovascularisation. However, within impaired wound healing, decreased VEGF expression is observed, which is credited to DNA damage and lipid peroxidation induced by the wound environment. Interestingly, OL has been shown to increase VEGF expression during in vivo wound healing studies, which was correlated with the observed acceleration of re-epithelialisation. Thus, olive phenols may serve as suitable pro-angiogenic compounds in the context of inflammatory environments that are associated with wound healing.

Mitochondria account for up to 30% of the total cell volume, and notably, are the only sites where extra-nuclear DNA resides. More significantly, mitochondria consume around 90% of the cell's oxygen and are the richest source of reactive oxygen species (ROS). Perturbations in mitochondrial function may therefore significantly contribute to disturbances in oxidation-reduction reactions that determine the cellular redox environment. The mitochondrion serves as the ‘power house’ of the cell, through oxidative phosphorylation (OXPHOS). It is the cells principal mechanism of energy production that incorporates the tricarboxylic acid (TCA) cycle and electron transport chain (ETC). The TCA cycle occurs within the mitochondrial matrix, and works to unify the metabolism of carbohydrates, lipids and amino acids, which integrate into the TCA cycle to produce the electron-rich donors to be utilized within the ETC. This process begins with acetyl-CoA, which is derived from the catabolism of sugars, fats and proteins, undergoes complete oxidation into two molecules of CO₂. The reactions of the TCA cycle also reduce nicotinamide adenine dinucleotide (NAD⁺) and flavin adenine dinucleotide (FAD) into electron rich donors NADH and FADH₂. These essential intermediates enter the ETC on the mitochondrial inner membrane for use in adenosine triphosphate (ATP) production. The ETC is composed of five protein complexes (I-V) that perform a series of redox reactions where O2 serves as the final electron acceptor and is reduced to H₂O. Electron transfer across the protein complexes is coupled with the ejection of H⁺ into the intermembrane space, creating a proton gradient that drives the production of ATP through OXPHOS.

Methods for improved wound closure employing olive phenolics, such as oleuropein (OL) and hydroxytyrosol (HT) are described, for example, in U.S. patent application Ser. No. 14/328,843, which is incorporated herein in its entirety. However, a better understanding of the effects of olive phenolics on angiogenic capacity human microvascular endothelial cells (HMEC-1) and human umbilical vein endothelial cells (HUVEC) allows for the development of better therapeutics. Further, enhanced formulations with desirable bioenergetic effects will provide superior therapeutic, prophylactic, and/or maintenance treatments and products.

Accordingly, it is an object of the invention to provide methods for wound healing including providing a patient with a combination of OLIVAMINE® (compositions of hydroxytyrosol and oleuropein) composition and a source of endothelial cells for improved treatment over conventional stem cell transplantation alone.

It is further objective of the claimed invention to significantly accelerate wound closure and/or promote neurovascularization of tissues, including treatment of wounds that conventionally resist healing.

A further objective of the claimed invention is to provide compositions and methods to enhance the angiogenic capacity of endothelial cells, including microvascular and umbilical vein endothelial cells.

A further objective of the claimed invention is to provide compositions and methods to enhance the bioenergetic characteristics of cells and tissues.

A further object of the invention is to develop treatment methods for substantially decreasing time for wound repair post injury, in some aspects providing beneficial results in 12 hours post injury or even 8 hours post injury, or less.

These and other objects and aspects of the invention are set forth herein the description of the invention.

### BRIEF SUMMARY OF THE INVENTION

The methods of the invention overcome a significant limitation of the art of wound healing; namely, compositions of hydroxytyrosol and oleuropein are provided with human umbilical vein endothelial cells to significantly reduce the required time for healing a wound. Although umbilical vein endothelial cells are known to provide benefits for wound healing, the methods of the present invention synergistically enhance wound repair.

In an aspect of the invention, a method for accelerating wound closure to improve wound healing is provided. In an aspect, the method includes administering or transplanting to a subject in need of wound healing a composition comprising an effective amount of hydroxytyrosol and oleuropein and an effective amount of endothelial cells, wherein the administration of the composition reduces the time required for healing of the wound by at least about 30% in comparison to a composition treated with the endothelial cells alone.

In a further aspect of the invention, a method for promoting cellular migration to improve wound healing is provided. In an aspect, the method includes topically administering or transplanting to a subject in need of wound healing a composition comprising hydroxytyrosol and oleuropein and endothelial cells, and reducing the time required for wound healing at least 50% of a wound, as measured by cellular migration to close a wound, to below at least 12 hours.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1(A-C) shows wound healing time is decreased when treated with the present invention. (A) shows comparison of wounds left untreated and treated with the present invention over 48 hours. (B) shows quantification of wound healing over time either without any treatment or with treatment using the present invention. Wound healing was accelerated by treatment with the present invention at all time-points investigated. (C) Quantification of the time it takes a wound to heal by 50% in the presence or absence of treatment with the present invention. Wound healing time was synergistically improved with treatment.

FIG. 2 shows compositions according to the invention improve wound healing both in the presence and absence of glucose.

FIG. 3(A-C) shows impaired wound healing in the presence of high glucose concentrations is normalized by treatment with the composition of the present invention. (A) shows comparison of wound healing in the presence of the combination of hydroxytyrosol and N-acetyl-L-cysteine of the present invention with high glucose (HT+NALC+glucose), in the presence of high glucose alone, or untreated. (B) shows quantification of wound healing in panel A. The HT+NALC combination maintains healthy wound healing times in a high glucose environment (C) shows comparison of the three treatments for the time it takes the wound to heal by 50%.

FIG. 4 shows the hydrolysis of oleuropein (25% is composed of hydroxytyrosol) into hydroxytyrosol.

FIG. 5 shows various photographs of a 7 day wound blind study of wound healing using a combination of hydroxytyrosol and N-acetyl-L-cysteine, wherein wound No. 2 shows significantly better wound healing than other wound sites.

FIG. 6(A-B) show exemplary images of measurements of the distance between wound openings at varying points of time, pursuant to Example 2 of the Application.

FIG. 7(A-B) shows wound healing at 0 and 24 hours according to an exemplary embodiment of the invention. (A) shows images of various wounds at zero hours and 24 hours post-wound. (B) shows graphically the healing (e.g. decrease in percentage of open wound) according to an untreated control, a wound administered hydroxytyrosol and a wound administered OLIVAMINE® (compositions of hydroxytyrosol and oleuropein) composition according to an embodiment of the invention.

FIG. 8(A-B) shows wound healing at 0 and 24 hours according to an exemplary embodiment of the invention. (A) shows images of various wounds at zero hours and 24 hours post-wound under high glucose conditions. (B) shows graphically the healing (e.g. decrease in percentage of open wound) according to an untreated control, a wound administered hydroxytyrosol and a wound administered OLIVAMINE® (compositions of hydroxytyrosol and oleuropein) composition according to an embodiment of the invention.

FIG. 9(A-F) show graphs of the tested effects of the individual components of the hydroxytyrosol and oleuropein compositions on the growth of a vascularized endothelial cell line according to embodiments of the invention.

FIG. 10(A-B) shows a graph of the tested effects of double component combinations of the hydroxytyrosol and oleuropein compositions on the growth of a vascularized endothelial cell line according to embodiments of the invention.

FIG. 11 shows a graph of the tested effects of triple component combinations of the hydroxytyrosol and oleuropein compositions on the growth of a vascularized endothelial cell line according to embodiments of the invention.

FIG. 12 shows a graph of the tested effects of four-way component combinations of the hydroxytyrosol and oleuropein compositions on the growth of a vascularized endothelial cell line according to embodiments of the invention.

FIG. 13 shows a graph of the tested effects of five-way component combinations of the hydroxytyrosol and oleuropein compositions on the growth of a vascularized endothelial cell line according to embodiments of the invention.

FIG. 14 shows a graph of the compositions according to the invention on the relative cell viability of a vascularized endothelial cell line according to embodiments of the invention.

FIG. 15 shows a graph of various tested formulations to show the impact on relative cell viability of a vascularized endothelial cell line according to embodiments of the invention.

FIG. 16-17 shows a graph of various tested formulations to show the impact on the average γH2AX foci per cell according to embodiments of the invention for wound healing.

FIG. 18 shows a graph of the compositions according to the invention on the relative cell viability of a vascularized endothelial cell line according to embodiments of the invention.

FIG. 19 shows increased tube formation in vascular endothelial cells in response to treatment with an exemplary formulation. HMEC-1 and HUVEC cells were treated with Formulation 1 (Table 8, dilution factor=4; see Example 3) for 24 hours prior to conducting angiogenesis assays. Data presented as the percentage area tube formation at 6 hours compared to 0 hours. Shown are the mean±standard error of the mean (SEM) of two independent experiments performed in triplicate.

FIG. 20 shows no significant effects on cell viability in vascular endothelial cells. HMEC-1 cells were incubated with the indicated treatment for 24 hours (HT: 0-200 μM, OL: 0-800 μM, OLV (Formulation 1, 0-4×; see Table 8, Example 3) and cell viability was measured by crystal violet absorption. Data is presented as the of percentage growth inhibition relative to the untreated cells. Shown are the mean±SEM of three independent experiments.

FIG. 21(A-F) show responses to mitochondrial stress in vascular endothelial cells and peripheral circulating monocytes (PBMCs). HMEC-1 cells or PBMCs were treated with and without Formulation 1 (dilution factor=4) for 24 hours prior to performing a mito stress test using the SEAHORSE EXTRACELLULAR FLUX ANALYSER® system. Oxygen consumption rates (OCR) was measured before and after sequential injections of 1 μM oligomycin (ATP-synthase inhibitor), Carbonyl cyanide-4-(trifluoromethoxy)phenylhydrazone (FCCP) (proton gradient uncoupler), and antimycin A/rotenone (complex I inhibitor). The indicated parameters were determined in excel using the appropriate formula. Shown are the mean±SEM of two independent experiments performed up to six replicates.

FIG. 22(A-C) shows oxygen consumption rates in unstimulated PBMCs (A), PBMCs stimulated with 25 ng/mL PMA (B) and HMEC-1 (C) pre-treated with or without Formulation) (dilution factor 4) for 24 hours as measured by the SEAHORSE EXTRACELLULAR FLUX ANALYSER® system. Data shown represents the mean±SEM of one independent experiment performed in triplicate. A total of 3 independent experiments performed.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION OF THE INVENTION

The embodiments of this invention are not limited to particular methods and/or compositions for treating wounds, which can vary and are understood by skilled artisans. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers defining the range and include each integer within the defined range.

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

In the present invention, an “effective amount” or “therapeutically effective amount” of a compound or of a composition of the present invention is that amount of such compound and/or composition that is sufficient to effect beneficial or desired results as described herein. In terms of treatment of a mammal, e.g., a human patient, an “effective amount” is an amount sufficient to treat, reduce, manage, palliate, ameliorate, or stabilize a condition, such as a non-congenital oncosis or extended quiescence of the cells of a mammal, or both, as compared to the absence of the compound or composition.

As used herein, the term “stem cell” refers to a master cell that can reproduce indefinitely to form the specialized cells of tissues and organs. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells. As used herein, the term “pluripotent cell” refers to a cell that has complete differentiation versatility, i.e., the capacity to grow into any of the mammalian body's approximately 260 cell types. A pluripotent cell can be self-renewing, and can remain dormant or quiescent within a tissue. Unlike a totipotent cell (e.g., a fertilized, diploid egg cell), an embryonic stem cell cannot usually form a new blastocyst. As used herein, the term “multipotent cell” refers to a cell that has the capacity to grow into any of subset of the mammalian body's approximately 260 cell types. Unlike a pluripotent cell, a multipotent cell does not have the capacity to form all of the cell types.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

Wound Treatment

Surprisingly, it has been discovered that hydroxytyrosol, a potent anti-oxidant, can influence whether a cell is in a quiescent or proliferative state. More specifically, when present at a concentration above a threshold level, hydroxytyrosol can induce proliferative cells into a quiescent state and help maintain cells in a pre-existing quiescent state. Based upon evidence obtained to-date, the threshold concentration is about 10 μM hydroxytyrosol.

Still further, it has surprisingly been discovered that a hydroxytyrosol and oleuropein combination provides further benefits for wound healing. The combination of hydroxytyrosol and oleuropein along with additional nutritional supplement components of N-acetyl cysteine, glycine, L-taurine, L-proline and optional additional components provide further improved wound healing through the induction of proliferative cells. In addition, the administration of the hydroxytyrosol and oleuropein compositions with endothelial cells provides even further improvements in wound healing. Exemplary compositions containing hydroxytyrosol and oleuropein are commercially-available under the tradename OLIVAMINE® (compositions of hydroxytyrosol and oleuropein) composition (available from Pinnaclife®), such as those disclosed in related application Ser. No. 12/853,908, which is herein incorporated by reference in its entirety.

According to the invention, the delivery of endothelial cells and/or transplantation of endothelial cells in combination with the hydroxytyrosol and oleuropein compositions provides significantly improved wound healing. As referred to herein, the use of endothelial cells preferably includes the use of human umbilical cord blood stem cells. The cells may also include and be referred to as umbilical vein endothelial cells (i.e. HUVEC), umbilical cord blood cells, nucleated cells derived from umbilical cord blood, or the like, which are understood to refer to cells derived from the endothelium of veins, including but not limited to the endothelium of veins in the umbilical cord.

The umbilical endothelial cells according to the invention may be isolated and extracted form a healthy source, such as human blood and/or a blood source that is induced in an animal model. In some aspects, the blood or blood source is cord blood. In some aspects, the cells are harvested from term and/or pre-term deliveries and cultured thereafter. There are various known methods of isolating these cells, including for example, a modified Ficoll-Hypaque method, a 3% gelatin method, and/or a Ficoll-Hypaque method (Kim et al., Optimal umbilical cord blood processing: Basic study for the establishment of cord blood bank, Korean Journal of Hematopoietic Stem Cell Transplantation. 2000.5:61-68). The endothelial cells, in other aspects, may be obtained from commercial sources as one skilled in the art will ascertain, such as set forth in the Examples.

In still other aspects, the endothelial cells may be provided in the form of a medium conditioned by endothelial cells. In still other aspects, the growth factors which are beneficially increased as a result of an endothelial stem cell transplant (e.g. paracrine factors) may be directly administered in place of the endothelial cells themselves, such as disclosed by Kim et al., Cell Transplantation, Vol. 19:1635-1644 (2010), which is herein incorporated by reference in its entirety.

According to the invention, the cells may initially be incubated and/or cultured prior to administration according to the methods of the invention. Accordingly as used herein, the cells (or the growth factors) may be transplanted, infused or otherwise provided to a mammal, such as a human, in need of wound repair. In an aspect, a composition for in vivo transplantation of endothelial cells is provided for wound repair. In further aspects of the invention, a composition for in vivo transplantation of endothelial cells with a composition comprising hydroxytyrosol and oleuropein is provided for wound repair.

In a preferred aspect, the endothelial cells are transplanted into a wound in need of treatment; thereafter the wound and transplanted cells are contacted by the compositions comprising hydroxytyrosol and oleuropein. In a preferred aspect, the hydroxytyrosol and oleuropein composition is administered in the form of a gel, hydrogel and/or solution for covering the wound and transplanted endothelial cells. In a still further aspect, the composition may be contacted by means of a saturated cloth or other component that covers the wound. In still other aspects, the composition may be contacted by means of direct application to the wound and transplanted cells in need of improved wound healing.

In another aspect, the wound tissue (before transplantation of the umbilical vein endothelial cells) may initially be contacted by the compositions comprising hydroxytyrosol and oleuropein. In a preferred aspect, the hydroxytyrosol and oleuropein composition is administered to the wound bed as a means of a pretreatment, such as in the form of a gel, hydrogel and/or solution for contacting the wound bed. A covering may further be applied over the treated wound bed for a period of time. Thereafter, the umbilical vein endothelial cells are transplanted into the pre-treated wound bed.

According to aspects of the methods of the invention, wound repair includes the cell proliferation in a wound and/or the cellular migration toward wound healing. According to further aspects of the methods of the invention, wound repair includes stimulated keratinocyte and/or fibroblast proliferation at an earlier time than treatments with the endothelial cells alone. As referred to herein, “wound repair” refers to the time required for wound closure. In some aspects according to the invention, wound repair is significantly accelerated.

In some aspects of the invention, wound repair is observed as early as 3 days post injury with the combined treatment of hydroxytyrosol and oleuropein compositions with endothelial cells. In preferred aspects, wound repair is observed as early as 1 day post injury according to the methods of the invention. In still further preferred aspects, wound repair is observed as early as 12 hours post injury, or preferably 8 hours post injury according to the methods of the invention.

Without being limited to a particular mechanism of action according to the invention, the improved and accelerated wound healing according to the methods of the invention results from the synergistic effects of the EPCs derived from the endothelial cells secreting wound healing-related growth factors, along with the beneficial effects of the hydroxytyrosol and oleuropein compositions disclosed herein. In some aspects, the wound healing-related growth factors may include for example, keratinocyte growth factor and platelet-derived growth factor in the dermal tissue where the endothelial cells are transplanted. In a further aspect, paracrine factors from EPCs may directly exert mitogenic and chemotactic effects on keratinocytes and fibroblasts to further promote wound healing and increase neovascularization of the endothelial cells of the wound.

Beneficially, according to the invention, the use of umbilical endothelial cells with the hydroxytyrosol and oleuropein compositions of the invention provide improved wound healing and treatment methods in comparison to use of either the OLIVAMINE® (compositions of hydroxytyrosol and oleuropein) composition and/or the EPC transplantation into a wound alone (e.g. engraftment for vasculogenesis effects). In an aspect, the combination therapy according to the invention provides at least a 25% improvement over use of an endothelial cell transplant alone to repair a wound. In a further aspect, the combination therapy according to the invention provides at least a 30% improvement over use of an endothelial cell transplant alone to repair a wound. In still further preferred aspects, the combination therapy provides at least a 50% improvement or greater, and preferably at least a 60% improvement or greater.

Clinically the improved results disclosed according to the combination therapy according to the invention result in decreased time for wound healing. In some aspects the time required for healing at least 50% of a wound (as measured by cellular migration to close a wound) was reduced below 20 hours, preferably reduced below 15 hours, preferably reduced below 10 hours, and still more preferably reduced below about 8 hours. Beneficially, the rapid repair in wound healing according to the invention results from the combined use of the hydroxytyrosol and oleuropein compositions with the endothelial cells allowing the synergistic results of the improvement in cellular function and the growth factor and/or cytokine action elicited by the endothelial cells. This is particularly beneficial in wound repair in persons having underling pathophysiological abnormalities (e.g. diabetes) wherein would repair does not follow orderly progression as occurs in a healthy individual. As a result, the providing of endothelial cells is expected to overcome the reduced number of EPCs in the system of a person having such pathophysiological abnormalities.

The compositions described herein are preferably employed as topical compositions. They are preferably applied to the surface of the skin, mucosal cells and tissues (e.g., alveolar, buccal, lingual, masticatory, or nasal mucosa, and other tissues and cells that line hollow organs or body cavities) or exposed tissue.

Without being bound to any particular theory and based upon evidence obtained to-date, compositions of the present invention may be used to improve the health and viability of skin cells that are diseased or distressed as a result of a metabolic condition. For example, compositions comprising hydroxytyrosol and oleuropein may be used to reduce the concentration of free-radicals in the cells of skin tissue to improve cellular function. In addition, compositions comprising sufficient hydroxytyrosol and oleuropein may be used to induce cells into or maintain them in a reversible quiescent state to provide them with time to heal and return to a more viable state with a reduced risk of necrosis. In a preferred embodiment, such compositions further comprise N-acetyl cysteine.

In another preferred embodiment, such compositions additionally comprise N-acetyl cysteine. In another preferred embodiment, the composition further comprises hydroxytyrosol, N-acetyl cysteine and an additional component having a molecular weight not in excess of 500 Daltons that improves the health or viability of skin cells. Such additional components, for example, may be selected from the group consisting of glycine, L-taurine, L-proline, niacinamide (vitamin B3), pyridoxine (vitamin B6), methylsulfonylmethane, and combinations thereof.

The compositions and methods of the present invention may be used to treat skin that is dry, cracked, scaly, or exhibiting redness or edema but otherwise appears intact to the unaided eye. These symptoms may be presented as a result of an underlying disease or metabolic condition such as diabetes or, alternatively, may be caused by excessive transepidermal water loss. Transepidermal water loss in excess of about 5 g/hr/cm² can activate an inflammatory response in the epidermis and dermis. Many factors, such as relative humidity below 40%, changes in skin pH, normal aging and disruption of the stratum corneum contribute to excessive transepidermal water loss.

The compositions and methods of the present invention may be used to treat more serious wounds, that is, wounds characterized by a partial or total thickness skin loss, including wounds that are at risk of necrosis. When a wound is characterized by a partial or total thickness skin loss, one of the phases of wound healing is the proliferative phase. The proliferative phase typically includes angiogenesis, collagen deposition, granulation tissue formation, epithelialization, and wound contraction. Wound closure thus requires that cells be in a proliferative phase and it is preferred, therefore, that any composition applied to an open wound not induce the cells in the open wound area into a quiescent state.

According to methods of the invention wherein open wounds are treated, the use of the hydroxytyrosol and oleuropein compositions are combined with transplantation of umbilical vein endothelial cells.

In general, it is preferred that compositions applied to an open wound contain hydroxytyrosol and oleuropein in a concentration that is less than the threshold concentration at which quiescence is induced or maintained. Stated differently, it is generally preferred that compositions applied to Stage II, Stage III or Stage IV wounds contain hydroxytyrosol and oleuropein in a concentration that is less than the threshold concentration at which quiescence is induced or maintained. In one embodiment, therefore, compositions applied to an open or Stage II, III or IV wound contain hydroxytyrosol and oleuropein in a concentration not in excess of about 15 μM hydroxytyrosol and 56 μM oleuropein. For example, compositions applied to an open or Stage II, III or IV wound may contain hydroxytyrosol in a concentration of at least about 1 μM but not in excess of about 15 μM hydroxytyrosol. By way of further example, such compositions may contain hydroxytyrosol in a concentration of about 1 to about 12 μM. In certain embodiments, the concentration of hydroxytyrosol in such compositions will typically be between about 1 μM and 10 μM hydroxytyrosol. In other embodiments, the concentration of oleuropein in such compositions will typically be between about 4 μM and 60 μM oleuropein. As described herein, it is preferred that the ratio of oleuropein to hydroxytyrosol is from about 1:1 to about 10:1, preferably from about 2:1 to about 5:1, and most preferably in a ratio of about 4:1. Without being limited according to the invention all ranges within the ratios are further included within the scope of the invention.

In other regions, that is, regions appearing intact to the unaided eye such as (i) the peri-wound region surrounding an open wound, (ii) skin that is dry, cracked, scaly, or exhibiting redness or edema but otherwise appears intact to the unaided eye, or (iii) skin experiencing excessive transepidermal water loss but otherwise appears intact to the unaided eye may be treated with compositions containing hydroxytyrosol in a concentration that is greater than the concentration at which quiescence is induced or maintained. Stated differently, it is generally preferred that compositions applied to wounds not characterized by a partial or total thickness skin loss (Stage I or less severe wounds sometimes called Stage 0) contain hydroxytyrosol in a concentration that is greater than the threshold concentration at which quiescence is induced or maintained. In one embodiment, therefore, compositions applied to a Stage 0 or Stage I wound contain hydroxytyrosol in a concentration in excess of 5 μM but not in excess of about 250 μM hydroxytyrosol, and further contain oleuropein in a concentration in excess of 20 μM but not in excess of about 1000 μM oleuropein.

For example, compositions applied to (i) the peri-wound region surrounding an open wound, (ii) skin that is dry, cracked, scaly, or exhibiting redness or edema but otherwise appears intact to the unaided eye, or (iii) skin experiencing excessive transepidermal water loss but otherwise appears intact to the unaided eye may contain hydroxytyrosol in a concentration in excess of about 250 μM. In one embodiment, such compositions may contain hydroxytyrosol in a concentration of about 5 μM to about 250 μM, and an oleuropein concentration of about 20 μM to about 1000 μM. In certain embodiments, such compositions may contain hydroxytyrosol in a concentration of about 7 μM to about 225 μM, and oleuropein concentration of about 28 μM to about 900 μM. In certain embodiments, such compositions may contain hydroxytyrosol in a concentration of about 10 μM to about 200 μM, and oleuropein concentration of about 40 μM to about 800 μM. In certain embodiments, such compositions may contain hydroxytyrosol in a concentration of at least 15 μM but not in excess of 200 μM, and oleuropein concentration of at least about 60 μM but not in excess of 800 μM.

Treatment of a Stage II, III or IV wound preferably comprises treatment of the peri-wound region with a first composition and treatment of the wound region with a second composition wherein the first composition contains hydroxytyrosol in a concentration at which quiescence is induced or maintained and the second composition contains hydroxytyrosol in a concentration that is less than the concentration at which quiescence is induced or maintained. Typically, the two compositions will be applied 2 to 3 times daily at regularly spaced intervals until the wound has filled (i.e., closes); at that point, the first composition may be applied 2 to 3 times daily at regularly spaced intervals to the closed wound and the pen-wound region. Advantageously, application of the first composition to the closed wound will tend to reduce scarring. In one embodiment, the first composition will be applied to the closed wound for up to 18 months after closure of the wound without a recurrence of the wound.

Treatment of regions appearing intact to the unaided eye such as (i) the peri-wound region surrounding an open wound, (ii) skin that is dry, cracked, scaly, or exhibiting redness or edema but otherwise appearing intact to the unaided eye, or (iii) skin experiencing excessive transepidermal water loss but otherwise appearing intact to the unaided eye may be treated with compositions containing hydroxytyrosol in a concentration that is greater than the concentration at which quiescence is induced or maintained until the region is asymptomatic. For pen-wounds, the composition is preferably applied to the entire peri-wound region and adjacent skin within at least about 1 cm of the pen-wound region. Typically, the composition will be applied 2 to 3 times daily at regularly spaced intervals at least until the region is asymptomatic.

Compositions containing hydroxytyrosol in a concentration that is greater than the concentration at which quiescence is induced or maintained may also be applied prophylactically to regions that are perceived to be at risk of a chronic wound, such as venous ulcers and diabetic ulcers. In one embodiment, chronic wounds are treated using a composition of the present invention to help reverse the damage to the cells in the wound and pen-wound areas and inhibit necrosis. For example, such compositions may be applied to regions in which there are symptoms of neuropathy or lack of capillary integrity. By way of further example, such compositions may be applied to the lower leg, e.g., from the knee to the tips of the toes.

Compositions According to the Invention

In an aspect of the invention, the compositions include hydroxytyrosol or an ester or salt thereof and oleuropein. In an aspect the ratio of hydroxytyrosol to oleuropein is from about 1:1:to about 1:10, in a preferred aspect, the ratio of hydroxytyrosol to oleuropein is from about 1:2:to about 1:8, preferably about 1:4. Without being limited according to the invention all ranges within the ratios are further included within the scope of the invention.

In addition to hydroxytyrosol and oleuropein, the topical compositions of the present invention may contain N-acetyl cysteine and/or an additional component having a molecular weight not in excess of 500 Daltons that improves the health or viability of skin cells. Such additional components, for example, may include other antioxidants, vitamins, minerals, and/or amino acids. Non-limiting examples of other antioxidants include ascorbic acid (vitamin C) and its salts, ascorbyl esters of fatty acids, ascorbic acid derivatives (e.g., magnesium ascorbyl phosphate, sodium ascorbyl phosphate, and ascorbyl sorbate), Epigallocatechin gallate (EGCG), oleuropein, tocopherol (vitamin E), tocopherol sorbate, tocopherol acetate, other esters of tocopherol, tyrosol, butylated hydroxy benzoic acids and their salts, gallic acid and its alkyl esters such as propyl gallate, uric acid and its salts and alkyl esters, sorbic acid and its salts, lipoic acid, amines (e.g., N,N-diethylhydroxylamine and amino-guanidine), sulfhydryl compounds (e.g., glutathione), dihydroxy fumaric acid and it salts, glycine pidolate, arginine pilolate, nordihydroguaiaretic acid, bioflavinoids, curcumin, lyseine, methionine, proline, superoxide dismutase, resveratrol, and other polyphenols. In another embodiment, the composition comprises hydroxytyrosol, N-acetyl cysteine, and one or more of cystine, cystine derivatives, vitamin C, tannic acid, vitamin E, vitamin E derivatives, catechin, niacin, unsaturated fatty acids, vitamin P, vitamin Q, glutathione, isoflavones, guava, selenium, oleuropein or other polyphenol(s). In one embodiment, the composition comprises hydroxytyrosol, N-acetyl cysteine and one or more of glycine, L-taurine, L-proline, niacinamide (vitamin B3), pyridoxine (vitamin B6), and methylsulfonylmethane.

In one embodiment, the composition contains non-amino acid additives such as aloe vera, oat extract, hyaluronic acid, betaglucan or like substance to provide glycosaminoglycans for extracellular matrix protection. Vitamins may be additives, especially vitamins A/D3, all B vitamins and all stable C vitamins. Omega 3 and 6 fatty acids will be balanced with the greater percentage being 3. In one embodiment, the composition may contain other antioxidants, anti-inflammatory agents and tissue repair ingredients known to have wound healing benefits. For example, in one embodiment, the composition contains olive leaf extract, vitamin A/D3, Vitamin C, and essential fatty acids from olive oil, canola oil, safflower oil, borrage oil and sunflower oil. Also preferably, olive leaf extract is present in the composition of the present invention.

In one embodiment, the composition contains N-acetyl cysteine and hydroxytyrosol and the weight ratio of N-acetyl cysteine to hydroxytyrosol to between 1:1 and 50:1, respectively. In one embodiment, the composition contains N-acetyl cysteine and hydroxytyrosol and the weight ratio of N-acetyl cysteine to hydroxytyrosol is between 10:1 and 30:1, respectively. For example, in one such embodiment, the composition contains N-acetyl cysteine and hydroxytyrosol and the weight ratio of N-acetyl cysteine to hydroxytyrosol is between 20:1 and 25:1, respectively.

In one embodiment, the composition contains glycine and hydroxytyrosol and the weight ratio of glycine to hydroxytyrosol to between 1:1 and 50:1, respectively. In one embodiment, the composition contains glycine and hydroxytyrosol and the weight ratio of glycine to hydroxytyrosol is between 30:1 and 40:1, respectively. For example, in one such embodiment, the composition contains glycine and hydroxytyrosol and the weight ratio of glycine to hydroxytyrosol is about 35:1, respectively.

In one embodiment, the composition contains L-taurine and hydroxytyrosol and the weight ratio of L-taurine to hydroxytyrosol to between 1:1 and 50:1, respectively. In one embodiment, the composition contains L-taurine and hydroxytyrosol and the weight ratio of L-taurine to hydroxytyrosol is between 20:1 and 50:1, respectively. In one embodiment, the composition contains L-taurine and hydroxytyrosol and the weight ratio of L-taurine to hydroxytyrosol is between 30:1 and 40:1, respectively. For example, in one such embodiment, the composition contains L-taurine and hydroxytyrosol and the weight ratio of L-taurine to hydroxytyrosol is about 35:1, respectively.

In one embodiment, the composition contains L-proline and hydroxytyrosol and the weight ratio of L-proline to hydroxytyrosol to between 1:1 and 20:1, respectively. In one embodiment, the composition contains L-proline and hydroxytyrosol and the weight ratio of L-proline to hydroxytyrosol is between 1:1 and 10:1, respectively. In one embodiment, the composition contains L-proline and hydroxytyrosol and the weight ratio of L-proline to hydroxytyrosol is between 1:1 and 5:1, respectively.

In one embodiment, the composition contains methylsulfonylmethane and hydroxytyrosol and the weight ratio of methylsulfonylmethane to hydroxytyrosol to between 1:1 and 30:1, respectively. In one embodiment, the composition contains methylsulfonylmethane and hydroxytyrosol and the weight ratio of methylsulfonylmethane to hydroxytyrosol is between 5:1 and 25:1, respectively. In one embodiment, the composition contains methylsulfonylmethane and hydroxytyrosol and the weight ratio of methylsulfonylmethane to hydroxytyrosol is between 10:1 and 20:1, respectively.

In one embodiment, the composition contains niacinamide and hydroxytyrosol and the weight ratio of niacinamide to hydroxytyrosol to between 1:1 and 10:1, respectively. In one embodiment, the composition contains niacinamide and hydroxytyrosol and the weight ratio of niacinamide to hydroxytyrosol is between 1:1 and 5:1, respectively. In one embodiment, the composition contains niacinamide and hydroxytyrosol and the weight ratio of niacinamide to hydroxytyrosol is between 1:1 and 2:1, respectively.

In one embodiment, the composition contains pyridoxine and hydroxytyrosol and the weight ratio of pyridoxine to hydroxytyrosol to between 1:1 and 10:1, respectively. In one embodiment, the composition contains pyridoxine and hydroxytyrosol and the weight ratio of pyridoxine to hydroxytyrosol is between 1:1 and 5:1, respectively. In one embodiment, the composition contains pyridoxine and hydroxytyrosol and the weight ratio of pyridoxine to hydroxytyrosol is between 1:1 and 2:1, respectively.

In one preferred embodiment, the composition of the present invention contains hydroxytyrosol, N-acetyl cysteine and optionally one or more of glycine, L-taurine, L-proline, niacinamide (B3), pyridoxine (B6), and methylsulfonylmethane. In one example of this embodiment, the weight ratio N-acetyl cysteine to hydroxytyrosol is between 1:1 and 50:1, respectively, the weight ratio glycine to hydroxytyrosol is between 1:1 and 50:1, respectively, the weight ratio of L-taurine to hydroxytyrosol is between 1:1 and 50:1, respectively, the weight ratio of L-proline to hydroxytyrosol is between 1:1 and 20:1, respectively, the weight ratio of niacinamide to hydroxytyrosol is between 1:1 and 10:1, respectively, the weight ratio of pyridoxine to hydroxytyrosol is between 1:1 and 10:1, and the weight ratio of methylsulfonylmethane to hydroxytyrosol is between 1:1 and 30:1. In another example of this embodiment, the weight ratio N-acetyl cysteine to hydroxytyrosol is between 10:1 and 30:1, respectively, the weight ratio glycine to hydroxytyrosol is between 30:1 and 40:1, respectively, the weight ratio of L-taurine to hydroxytyrosol is between 20:1 and 50:1, respectively, the weight ratio of L-proline to hydroxytyrosol is between 1:1 and 10:1, respectively, the weight ratio of niacinamide to hydroxytyrosol is between 1:1 and 5:1, respectively, the weight ratio of pyridoxine to hydroxytyrosol is between 1:1 and 5:1, and the weight ratio of methylsulfonylmethane to hydroxytyrosol is between 10:1 and 30:1. In another example of this embodiment, the weight ratio N-acetyl cysteine to hydroxytyrosol is between 20:1 and 25:1, respectively, the weight ratio glycine to hydroxytyrosol is between 30:1 and 40:1, respectively, the weight ratio of L-taurine to hydroxytyrosol is between 30:1 and 40:1, respectively, the weight ratio of L-proline to hydroxytyrosol is between 1:1 and 5:1, respectively, the weight ratio of niacinamide to hydroxytyrosol is between 1:1 and 2:1, respectively, the weight ratio of pyridoxine to hydroxytyrosol is between 1:1 and 2:1, and the weight ratio of methylsulfonylmethane to hydroxytyrosol is between 10:1 and 20:1.

In each of the aforementioned embodiments, the components of the composition of the present invention may optionally be present in the form of an ester or a physiologically/pharmaceutically acceptable salt. Exemplary esters include the mono-, di- and triesters of hydroxytyrosol with (un)saturated carbonic acids R—COOH, whereby R is an alkyl or alkenyl chain having 2 to 22 carbon atoms. Exemplary pharmaceutically acceptable salts refer to salts prepared from pharmaceutically acceptable non-toxic acids, including inorganic salts and organic salts. Suitable non-organic salts include inorganic and organic acids such as acetic, benzene sulfonic, benzoic, camphor sulfonic, citric, ethane sulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluene sulfonic and other pharmaceutically acceptable salts as provided in Stahl and Wermuth “Pharmaceutical Salts Properties, Selection, and Use”, 1st Ed, Wiley-VCH, 374 (2002), which is herein incorporated by reference. Thus, for example, the term “hydroxytyrosol” also encompasses pharmaceutically acceptable salts thereof such as the sodium or potassium salts, or others of the aforementioned salts, or an ester thereof.

For use in the composition of the present invention, hydroxytyrosol and oleuropein may be derived from natural sources or prepared by chemical synthesis. For example, the hydroxytyrosol and oleuropein may be obtained as an extract of, or otherwise derived from, olive leaves, olive fruits and vegetation water of olive oil production. When obtained as an extract, for example, of olive leaves, the extract will contain hydroxytyrosol, tyrosol, oleuropein, and other polyphenols. In one preferred embodiment, the hydroxytyrosol is obtained as an olive leaf extract of Olea europaea.

The composition may be in any form suitable for application to the body surface, and may comprise, for example, a cream, lotion, solution, suspension, emulsion, gel, ointment, paste, or the like, and/or may be prepared so as to contain liposomes, micelles, and/or microspheres. In certain embodiments, it is preferred, although not essential, that water be present. Thus, such a formulation may be aqueous, i.e., contain water, or, alternatively, may be non-aqueous. Where the formulation is non-aqueous, it may be optionally used in combination with an occlusive overlayer so that moisture evaporating from the body surface is maintained within the formulation upon application to the body surface and thereafter. In one preferred embodiment, the formulation is aqueous.

The principal differences between the physical dose forms noted above (e.g., creams, lotions, gels, and aqueous liquids) are their physical appearance and viscosity (or thickness), which are governed primarily by the presence and amount of emulsifiers and viscosity adjusters; the main ingredients are, in many cases, common among these product forms. Moreover, a particular topical formulation may often be prepared in a variety of these forms. Ointments, creams and lotions are often similar to one another, differing mainly in their viscosity (creams are typically thicker and more viscous than lotions); both lotions and creams may be opaque, translucent or clear and often contain emulsifiers, solvents (including water and alcohol) and viscosity adjusting agents. Ointments, creams and lotions also may optionally contain moisturizers and emollients, as well as fragrances, dyes/colorants, preservatives and active ingredients. Gels may be prepared with a range of viscosities, from thick (high viscosity) to thin (low viscosity) and differ principally from lotions and creams in that gels are often (but not exclusively) clear rather than opaque. Like lotions and creams, gels often contain emulsifiers, solvents (including water and alcohol) and viscosity adjusters, and may also contain moisturizers and emollients, fragrances, dyes/colorants, preservatives and active ingredients. Aqueous liquids are thinner than creams, lotions or gels, and are generally transparent; liquids usually do not contain emulsifiers. Liquid topical products often contain other solvents in addition to water (including alcohol) and may also contain viscosity adjusters, moisturizers and emollients, fragrances, dyes/colorants/pigments, preservatives and active ingredients.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and non-sensitizing. Ointment bases may be grouped in four classes: oleaginous bases; emulsifiable bases; emulsion bases; and water-soluble bases (see, e.g., Remington: The Science and Practice of Pharmacy, 19th Ed. (Easton, Pa.: Mack Publishing Co., 1995), pages 1399-1404). Oleaginous ointment bases include, for example, vegetable oils, fats obtained from animals, and semisolid hydrocarbons obtained from petroleum. Emulsifiable ointment bases, also known as absorbent ointment bases, typically contain little or no water and include, for example, hydroxystearin sulfate, anhydrous lanolin, and hydrophilic petrolatum. Emulsion ointment bases are generally either water-in-oil (W/O) emulsions or oil-in-water (O/W) emulsions, and include, for example, cetyl alcohol, glyceryl monostearate, lanolin, and stearic acid. Certain preferred water-soluble ointment bases are generally prepared from polyethylene glycols of varying molecular weight.

Creams, as also well known in the art, are viscous liquids or semisolid emulsions, typically either oil-in-water or water-in-oil. Cream bases are water-washable, and typically contain an aqueous phase, an oil phase, and an emulsifier. The aqueous phase (e.g., water), usually, although not necessarily, exceeds the oil phase in volume, and generally contains a humectant. The oil phase is generally comprised of petrolatum and a fatty alcohol such as cetyl or stearyl alcohol. The emulsifier in a cream formulation is generally a nonionic, anionic, cationic, or amphoteric surfactant.

Lotions are preparations to be applied to the skin surface without substantial friction, and are typically liquid or semiliquid preparations in which the active agent is present in a water or alcohol base. Lotions may also be suspensions of solids, and may comprise a liquid oily emulsion of the oil-in-water type. In certain embodiments, lotions may be preferred for treating larger body areas, because of the ease of applying a more fluid composition. Lotions will typically contain suspending agents to produce better dispersions as well as compounds useful for localizing and holding the active agent in contact with the skin, e.g., methylcellulose, sodium carboxymethylcellulose, or the like.

Gels employed in the field of pharmaceutical formulation are semisolid, suspension-type systems. Single-phase gels contain organic macromolecules distributed substantially uniformly throughout the carrier liquid, which is typically aqueous, but also, preferably, contains an alcohol and, optionally an oil. Preferred gelling agents, are cross-linked acrylic acid polymers such as the carbomer family of polymers, e.g., carboxypolyalkylenes that may be obtained commercially (e.g., CARBOPOL® and the like). Other exemplary hydrophilic polymers include polyethylene oxides, polyoxyethylene-polyoxypropylene copolymers, and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, or stirring, or combinations thereof.

Pastes are semisolid dosage forms in which the active agent is suspended in a suitable base. Depending on the nature of the base, pastes may be divided between fatty pastes or those made from a single-phase aqueous gel. The base in a fatty paste is generally petrolatum, hydrophilic petrolatum, or the like. The pastes made from single-phase aqueous gels generally incorporate carboxymethylcellulose or the like as a base.

As noted above, topical formulations may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations may include cationic (positively charged), anionic (negatively charged), and neutral preparations. Cationic liposomes are readily available and include, for example, N[1-2,3-dioleyloxy)propyl]-N,N,N-triethylammonium (DOTMA) liposomes are available under the tradename LIPOFECTIN®. (GIBCO BRL, Grand Island, N.Y.). Similarly, anionic and neutral liposomes are readily available as well, e.g., from AVANTI POLAR LIPIDS, INC. (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphosphatidyl ethanolamine (DOPE), among others. These materials can also be mixed with DOTMA in appropriate ratios. Methods for making liposomes using these materials are well known in the art.

Micelles are known in the art as comprised of surfactant molecules arranged so that their polar headgroups form an outer spherical shell, while their hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10, and nonoxynol 30. Micelle formulations can be used in conjunction with the present disclosure either by incorporation into the reservoir of a topical or transdermal delivery system, or into a formulation to be applied to the body surface.

Microspheres, similarly, may be incorporated into the present topical formulations and drug delivery systems. Like liposomes and micelles, microspheres essentially encapsulate a drug or drug-containing formulation. Microspheres are generally, although not necessarily, formed from synthetic or naturally occurring biocompatible polymers, but may also be comprised of charged lipids such as phospholipids. Preparation of microspheres is well known in the art and described in the pertinent texts and literature.

The choice of a particular formulation carrier or vehicle will depend on the particular physical form and mode of delivery that the formulation is to achieve. Suitable topical vehicles and vehicle components for use with the formulations described herein (including, for example, the physical dose forms discussed above) are well known in the cosmetic and pharmaceutical arts, and include such vehicles (or vehicle components) and carriers as water; organic solvents such as alcohols (particularly lower alcohols readily capable of evaporating from the skin such as ethanol), glycols (such as propylene glycol, butylene glycol, and glycerin), aliphatic alcohols (such as lanolin); mixtures of water and organic solvents (such as water and alcohol), and mixtures of organic solvents such as alcohol and glycerin (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, demethiconol and dimethicone copolyol (DOW CORNING®); hydrocarbon-based materials such as petrolatum and squalane; anionic, cationic and amphoteric surfactants and soaps; sustained-release vehicles such as microsponges and polymer matrices; stabilizing and suspending agents; emulsifying agents; and other vehicles and vehicle components that are suitable for administration to the skin, as well as mixtures of topical vehicle components as identified above or otherwise known to the art. In one particular embodiment, the carrier or vehicle comprises water. The vehicle may further include components adapted to improve the stability or effectiveness of the applied formulation, such as preservatives, antioxidants, skin penetration enhancers, sustained release materials, and the like. Examples of such vehicles and vehicle components are well known in the art and are described in such reference works as Martindale—The Extra Pharmacopoeia (Pharmaceutical Press, London 1993) and Remington's Pharmaceutical Sciences.

In certain embodiments, the formulation includes a solvent. Suitable solvents for use in the formulations of the present invention include, but are not limited to, water, ethanol, butylene glycol, propylene glycol, isopropyl alcohol, isoprene glycol, glycerin, CARBOWAX™200 (compositions of polyethylene glycol 200), CARBOWAX™ 400 (compositions of polyethylene glycol 400), CARBOWAX™ 600 (compositions of polyethylene glycol 600), and CARBOWAX™ 800 (compositions of polyethylene glycol 800). In addition, combinations or mixtures of these solvents may be used according to the present invention. In one particular embodiment, the solvent is water.

Depending on the particular physical dose form, an emulsifier may be included. Suitable emulsifiers for use in the formulations described herein include, but are not limited to, INCROQUAT™ BEHENYL TMS (behentrimonium methosulfate, cetearyl alcohol), non-ionic emulsifiers like polyoxyethylene oleyl ether, PEG-40 stearate, ceteareth-12 (e.g., EMULGIN® B-1 manufactured by HENKEL), ceteareth-20 (e.g., EMULGIN® B-2 manufactured byHENKEL), ceteareth-30, LANETTE® 0 (manufactured by HENKEL; ceteareth alcohol), glyceryl stearate (e.g., CUTINA® GMS manufactured by HENKEL), PEG-100 stearate, Arlacel 165 (glyceryl stearate and PEG-100 stearate), steareth-2 and steareth-20, or combinations/mixtures thereof, as well as cationic emulsifiers like stearamidopropyl dimethylamine and behentrimonium methosulfate, or combinations/mixtures thereof. In addition, cationic emulsifiers may be combined or mixed with non-ionic emulsifiers.

Suitable viscosity adjusting agents (i.e., thickening and thinning agents) for the formulations described herein include, but are not limited to, protective colloids or non-ionic gums such as hydroxyethylcellulose (e.g., CELLOSIZE® HEC QP52,000-H, manufactured by AMERCHOL), xanthan gum, and sclerotium gum (AMIGEL® 1.0), as well as magnesium aluminum silicate (VEEGUM® ULTRA), silica, microcrystalline wax, beeswax, paraffin, and cetyl palmitate. In addition, appropriate combinations or mixtures of these viscosity adjusters may be utilized.

Suitable surfactants for use in the formulations of the present invention include, but are not limited to, nonionic surfactants like Surfactant 190 (dimethicone copolyol), Polysorbate 20 (TWEEN® 20), Polysorbate 40 (TWEEN® 40), Polysorbate 60 (TWEEN® 60), Polysorbate 80 (TWEEN® 80), lauramide DEA, cocamide DEA, and cocamide MEA, amphoteric surfactants like oleyl betaine and cocamidopropyl betaine (VELVETEX® BK-35), and cationic surfactants like PHOSPHOLIPID PTC (Cocamidopropyl phosphatidyl PG-dimonium chloride). Combinations of surfactants may also be employed.

The formulations may also include one or more preservatives. Suitable preservatives include, but are not limited to, anti-microbials such as GERMABEN® II (manufactured by ICI; propylene glycol, diazolidinyl urea, methylparaben, and propylparaben), methylparaben, propylparaben, imidazolidinyl urea, benzyl alcohol, sorbic acid, benzoic acid, sodium benzoate, dichlorobenzyl alcohol, and formaldehyde, as well as physical stabilizers and anti-oxidants such as alpha-tocopherol (vitamin E), sodium ascorbate/ascorbic acid, ascorbyl palmitate and propyl gallate. In addition, combinations or mixtures of these preservatives may also be used.

Various additives, known to those skilled in the art, may also be included in the topical formulations. In certain embodiments, for example, it may be desirable to include one or more skin permeation enhancers in the formulation. Examples of suitable enhancers include, but are not limited to, ethers such as diethylene glycol monoethyl ether (available commercially as TRANSCUTOL®) and diethylene glycol monomethyl ether; surfactants such as sodium laurate, sodium lauryl sulfate, cetyltrimethylammonium bromide, benzalkonium chloride, POLOXAMER® (231, 182, 184), TWEEN® (20, 40, 60, 80), and lecithin (U.S. Pat. No. 4,783,450); alcohols such as ethanol, propanol, octanol, benzyl alcohol, and the like; polyethylene glycol and esters thereof such as polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine, and triethanolamine; terpenes; alkanones; and organic acids, particularly citric acid and succinic acid. AZONE® and sulfoxides such as DMSO and C10 MSO may also be used.

Other enhancers are those lipophilic co-enhancers typically referred to as “plasticizing” enhancers, i.e., enhancers that have a molecular weight in the range of about 150 to 1000, and an aqueous solubility of less than about 1 wt. %. Lipophilic enhancers include fatty esters, fatty alcohols, and fatty ethers. Examples of specific fatty acid esters include methyl laurate, ethyl oleate, propylene glycol monolaurate, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, e.g., a C2-C4 alkane diol or triol, is substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995).

The formulations may also comprise one or more moisturizers. Suitable moisturizers for use in the formulations of the present disclosure include, but are not limited to, lactic acid and other hydroxy acids and their salts, glycerin, propylene glycol, butylene glycol, sodium PCA, CARBOWAX® 200, CARBOWAX® 400, and CARBOWAX® 800. Suitable emollients for use in the formulations described herein include, but are not limited to, PPG-15 stearyl ether, lanolin alcohol, lanolin, lanolin derivatives, cholesterol, petrolatum, isostearyl neopentanoate, octyl stearate, mineral oil, isocetyl stearate, Ceraphyl 424 (myristyl myristate), octyl dodecanol, dimethicone (DOW CORNING® 200-100 cps), phenyl trimethicone (DOW CORNING® 556), DOW CORNING® 1401 (cyclomethicone and dimethiconol), and cyclomethicone (DOW CORNING® 344), and MIGLYOL® 840 (manufactured by HULS; propylene glycol dicaprylate/dicaprate). In addition, appropriate combinations and mixtures of any of these moisturizing agents and emollients may be used in accordance with the present invention.

Suitable fragrances and colors, such as FD&C Red No. 40 and FD&C Yellow No. 5, may also be used in the formulations. Other examples of fragrances and colors suitable for use in topical products are known in the art.

Other suitable additional and adjunct ingredients which may be included in the formulations of the present invention include, but are not limited to, abrasives, absorbents, anti-caking agents, anti-foaming agents, anti-static agents, astringents (e.g., witch hazel, alcohol, and herbal extracts such as chamomile extract), binders/excipients, buffering agents, chelating agents (e.g., Versene EDTA), film forming agents, conditioning agents, opacifying agents, pH adjusters (e.g., citric acid and sodium hydroxide), and protectants. Examples of each of these ingredients, as well as examples of other suitable ingredients in topical product formulations, may be found in publications by The Cosmetic, Toiletry, and Fragrance Association (CTFA). See, e.g., CTFA Cosmetic Ingredient Handbook, 2nd edition, eds. John A. Wenninger and G. N. McEwen, Jr. (CTFA, 1992).

The formulations may also contain irritation-mitigating additives to minimize or eliminate the possibility of skin irritation or skin damage resulting from the pharmacologically active base or other components of the composition. Suitable irritation-mitigating additives include, for example: alpha-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N acetyl cysteine; cis-urocanic acid; capsaicin; and chloroquine. The irritant-mitigating additive, if present, may be incorporated into the present formulations at a concentration effective to mitigate irritation or skin damage.

In a preferred aspect of the invention, compositions may include endothelial cells. Preferably, the endothelial cells of the present invention are vascular endothelial cells. The endothelial cell may be umbilical cord blood stem cells, preferably human umbilical cord blood stem cells, umbilical vein endothelial cells, preferably human umbilical vein endothelial cells (HUVEC), microvascular endothelial cells, preferably human microvascular endothelial cells, umbilical cord blood cells, nucleated cells derived from umbilical cord blood, or the like, which are understood to refer to cells derived from the endothelium of veins, including but not limited to the endothelium of umbilical cord veins.

Endothelial cells according to the invention may be isolated and extracted from a healthy source, such as human blood and/or a blood source that is induced in an animal model. In some aspects, the blood or blood source may be cord blood. In some aspects, the cells are harvested from term and/or pre-term deliveries and cultured thereafter.

The endothelial cells, in other aspects, may be obtained from commercial sources as one skilled in the art will ascertain, such as set forth in the Examples.

In still other aspects, the endothelial cells may be provided in the form of a medium conditioned by endothelial cells. In still other aspects, the growth factors which are beneficially increased as a result of, for example, an umbilical cord blood stem cell transplant (e.g. paracrine factors) may be directly administered in place of the umbilical cord blood stem cells themselves

In a further preferred aspect of the invention, compositions include an effective amount of hydroxytyrosol, oleuropein, or a combination of hydroxytyrosol and oleuropein;

and an effective amount of endothelial cells. The inclusion of an effective amount of hydroxytyrosol, oleuropein, or a combination of hydroxytyrosol and oleuropein is described above. In some aspects, the endothelial cells are preferably human vascular endothelial cells. Such human vascular endothelial cells may include, for example, human microvascular endothelial cells. Such compositions including both (1) an effective amount of hydroxytyrosol, oleuropein, or a combination of hydroxytyrosol and oleuropein; and (2) endothelial cells can beneficially reduce the time required for healing of the wound by at least about 30% in comparison to a wound treated with the endothelial cells alone.

Administration of Compositions for Wound Healing

The compositions and formulations described herein can be administered as a pre-treatment (e.g. prior to a wound) such that cells are pretreated with the hydroxytyrosol and oleuropein prior to an injury or wound. In other embodiments, the compositions and formulations described herein are administered with the endothelial cells as a treatment to an existing wound.

The compositions and formulations described herein can be administered in accordance with a number of topical delivery routes and/or mechanisms. The method of delivery of the compositions may vary, but generally involves application of a formulation comprising hydroxytyrosol to an area of body surface affected with a wound, or the area surrounding such wound (i.e., the pen wound). For example, in one embodiment gels may be preferred for areas in which there is a partial or total loss of skin layers (Stage II, III or IV wounds) and ointments will be prepared for areas in which the skin appears to be intact to the unaided eye. Typical modes of delivery include application using the fingers; application using a physical applicator such as a cloth, tissue, swab, stick or brush (as achieved for example by soaking the applicator with the formulation just prior to application, or by applying or adhering a prepared applicator already containing the formulation—such as a treated or premoistened bandage, wipe, washcloth or stick—to the skin); spraying (including mist, aerosol or foam spraying); dropper application (as for example with ear drops); sprinkling (as with a suitable powder form of the formulation); and soaking. A gel, cream, ointment, or lotion, for example, may be spread on the affected surface and optionally gently rubbed in. A solution may be applied in like manner, but more typically will be applied with a dropper, swab, or the like, and carefully applied to the affected areas. Solutions may also be sprayed onto a surface using a spray applicator; being mixed with fibrin glue and applied (e.g., sprayed) onto a surface. In some embodiments, a composition of the present invention may be impregnated into absorptive materials, such as dressings, bandages, patches, and gauze, or coated onto the surface of solid phase materials, and placed on an affected area, with or without the use of gentle pressure and/or an adhesive material to secure the material to the area.

Other types and configurations of topically applied drug delivery systems may also be used in conjunction with the present invention, as will be appreciated by those skilled in the art of transdermal drug delivery. See, for example, Ghosh, Transdermal and Topical Drug Delivery Systems (Interpharm Press, 1997), particularly Chapters 2 and 8.

The dose regimen will depend on a number of factors that may readily be determined, such as severity of the affected region and responsiveness of the condition to be treated, but will normally be one or more doses per day, with a course of treatment lasting from several days to several months, or until a cure is effected or a diminution of disease state is achieved. One of ordinary skill may readily determine optimum dosages, dosing methodologies, and repetition rates. In general, it is contemplated that the formulation will be applied one to four times daily. With a skin patch, bandage, or dressing, the device is generally maintained in place on the body surface throughout a drug delivery period, typically in the range of 8 to 72 hours, and replaced as necessary.

The method of promoting cell health of the cells of a mammal is useful for, among other things, the treatment or prevention of skin ailments. Treatment of lymphedema-induced pruritis and of ichthyosis with an effective amount of the composition of the present invention is shown to treat or palliate the skin manifestations occurring in these disorders. Topical formulations were effective upon following the treatment regimen.

Without seeking to limit the invention or to be bound by any particular theory, it is believed that promoting or maintaining cell health of the cells of a mammal by administering a therapeutically effective amount of a composition of the present invention may act through one or more of the following mechanisms: a) treating or preventing oncosis or extended quiescence of the cells; b) maintaining or increasing the amount of adenosine triphosphate (ATP) in the extracellular spaces within a mammal; c) repairing the cell membranes within a mammal; d) restoring the normal osmotic balance across the cell membranes or stopping the flow of sodium ions in the cells; e) activating quiescent cells that have not moved normally through the cell cycle; 0 protecting against free radical damage to the cell, its organelles and the extracellular spaces; and g) protecting against cellular necrosis during the pre-lethal stages.

Having described the invention in detail, it will be apparent that modifications and variations are possible without departing from the scope of the invention defined in the appended claims. Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The contents of all references, patents, and patent applications cited throughout this application are hereby incorporated by reference. The invention is further illustrated by the following examples, which should not be construed as further limiting.

All publications and patent applications in this specification are indicative of the level of ordinary skill in the art to which this invention pertains. All publications and patent applications are herein incorporated by reference to the same extent as if each individual publication or patent application was specifically and individually indicated by reference.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

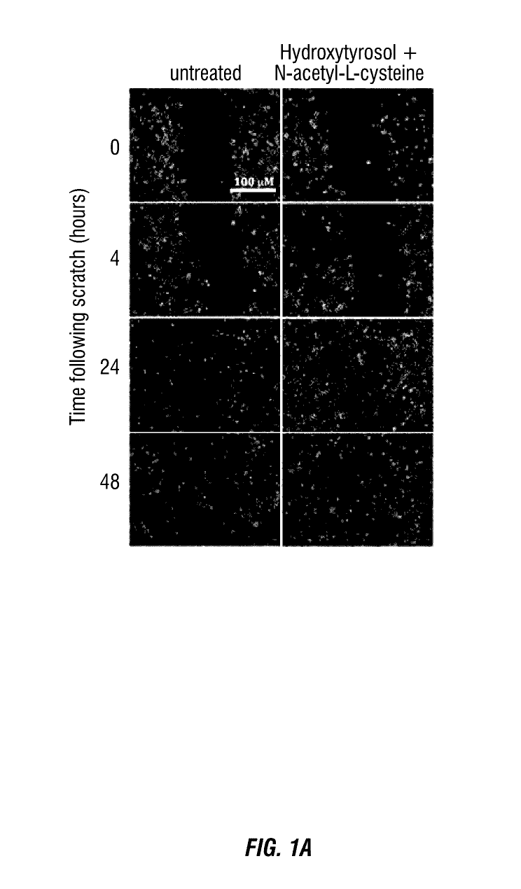

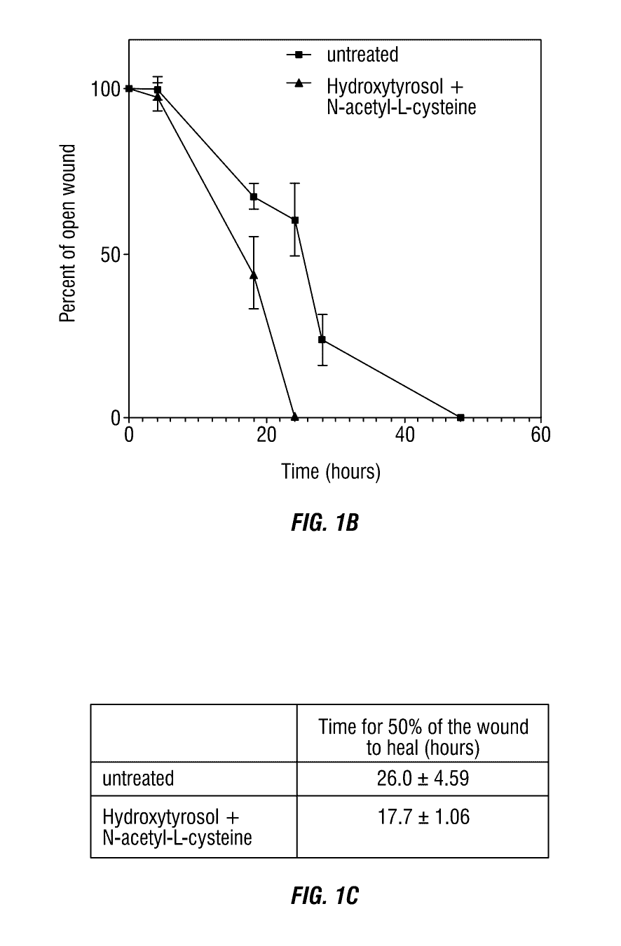

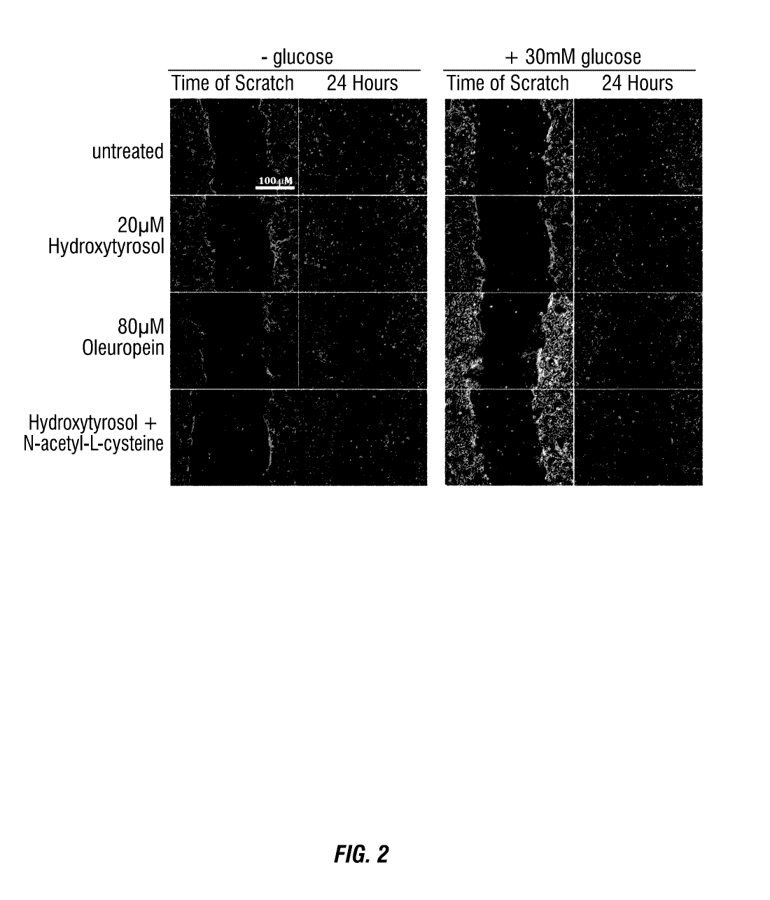

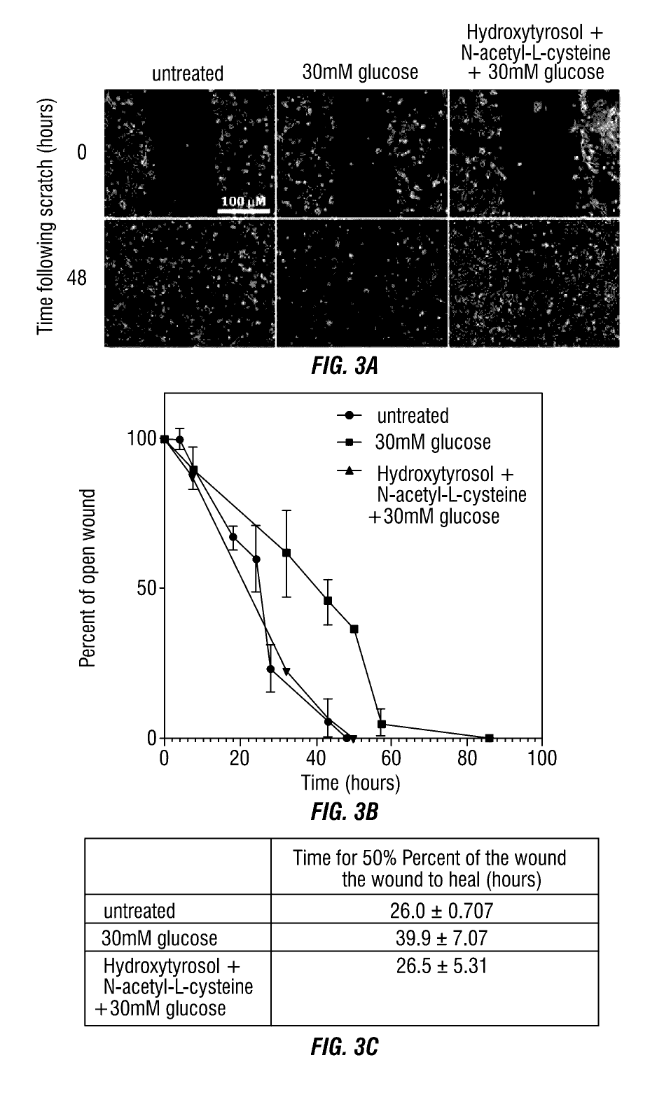

Original wound healing data evaluated the use of hydroxytyrosol and N-acetyl-L-cysteine for endothelial cell survival by 50%, and that in the presence of glucose, normal healing rates could be maintained by the combination. FIGS. 1-3 and Table 1 show the combination of hydroxytyrosol and N-acetyl-L-cysteine produced a synergistic effect on the time and extent of wound healing, both in the absence of glucose, and in the presence of glucose. The time for half of a wound to heal was shorter when both hydroxytyrosol and N-acetyl-cysteine were present than the additive effect of the compounds; N-acetyl-cysteine by itself increased the time to 50% healing compared to untreated. This is extremely significant and unexpected, primarily in light of the Warburg Effect, whereby high glucose levels impair wound healing.

TABLE 1

| | Time for 50% wound | |
|---|---|---|
| Treatment | to heal (hours) | SD |
| Untreated | 26.01 | 0.71 |
| Present Art | 17.75 | 1.06 |
| Hydroxytyrosol | 19.25 | 1.84 |
| Oleuropein | 25.62 | 2.17 |
| N-acetylcysteine | 28.87 | 1.95 |

As shown in Table 1, hydroxytyrosol and N-acetyl-L-cysteine work synergistically in wound healing. The time for wounds to heal by 50% under the various treatment conditions was quantified. Present Art is the combination of hydroxytyrosol and N-acetyl-L-cysteine of certain embodiments disclosed herein. The combination of hydroxytyrosol and N-acetyl-L-cysteine reduced healing times seen for each ingredient individually by up to 50%.

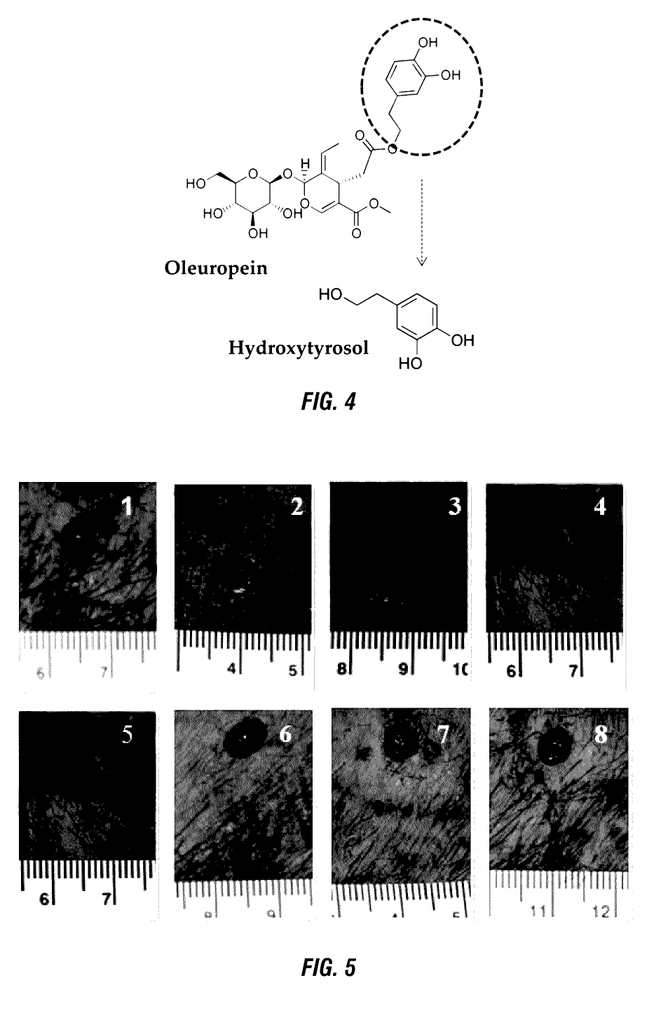

Additional research was conducted to demonstrate that the combination of hydroxytyrosol and N-acetyl-L-cysteine improves cell migration in an in vitro model of wound healing in human microvascular endothelial cells and improves wound healing by 50% as compared to untreated cells. In addition, oleuropein, of which 25% is composed of hydroxytyrosol and which is hydrolyzed into hydroxytyrosol (see FIG. 4), does not improve wound healing, and hydroxytyrosol alone is 30% less effective than when dosed at the concentrations anticipated by the present art (Table 1). Thus, the polyphenol oleuropein further including hydroxytyrosol allows for new treatment possibilities according to the embodiments of the invention.

This is demonstrated in FIG. 5 wherein treatments using the combination of hydroxytyrosol and N-acetyl-L-cysteine were provided topically in vivo. As shown in FIG. 5, wounds inflicted on a subject heal better when treated with the combination of hydroxytyrosol and N-acetyl-L-cysteine of the present invention.

Without being limited to a mechanism of the invention, the in vivo research has uncovered a heretofore unknown pathway that causes the cell to convert from anaerobic metabolism to aerobic energy production. The prior art did not contemplate the unique epigenetic interactions and mechanisms of olive phenolic compounds with the genome and the unique epigenetic interactions and mechanisms of olive phenolic compounds with the genome.

### Example 2

Additional wound studies were conducted using umbilical cord stem cells in addition to the use of hydroxytyrosol compositions and commercially-available OLIVAMINE® (compositions of hydroxytyrosol and oleuropein) compositions (20 μM hydroxytyrosol, 80 μM oleuropein, 2 mM N-acetylcysteine, 50 μM L-proline, 2 mM glycine and 100 μM taurine). These were evaluated with human umbilical vein endothelial cells (HUVEC) to assess impact on cell migration and wound healing.

HUVEC cells. Human umbilical vein endothelial cells are derived from the endothelium of veins from the umbilical cord (HUVEC primary cells purchased from Lonza). HUVECs have been widely used as a model system for the study of the regulation of endothelial cell function and the role of the endothelium in response of the blood vessel wall to stretch, shear forces and the development of atherosclerotic plaques and angiogenesis. For the following study, HUVECs are a primary endothelial cell line as a model system for the function and pathology of endothelial cells. We investigated improvement in cell proliferation and migration following treatment with OLIVAMINE® (compositions of hydroxytyrosol and oleuropein) composition.

In this study, HUVEC cells were pre-treated with or without 30 mM glucose for 3 days (72 hours). The cells were then equilibrated for a day (24 hours), and incubated with 50 μM hydroxytyrosol or OLIVAMINE® (compositions of hydroxytyrosol and oleuropein) formulation (20 μM hydroxytyrosol, 80 μM oleuropein, 2 mM N-acetylcysteine, 50 μM L-proline, 2 mM glycine and 100 μM taurine) for 24 hours before wounding.

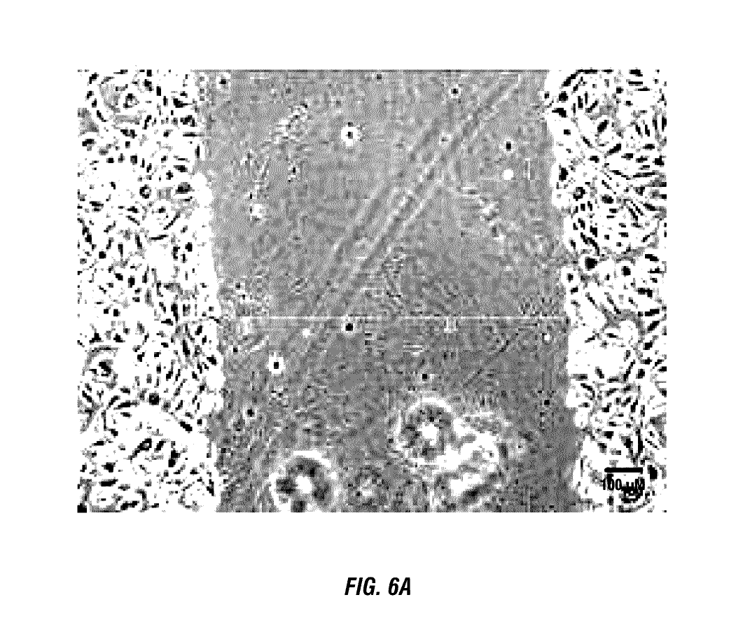

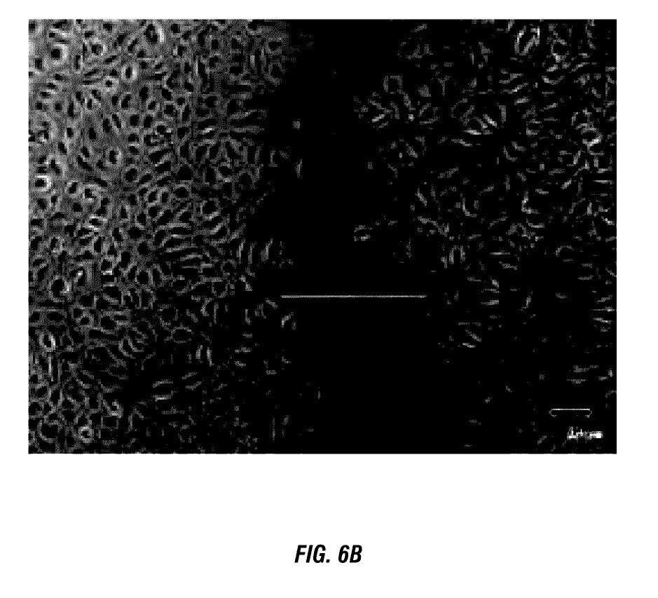

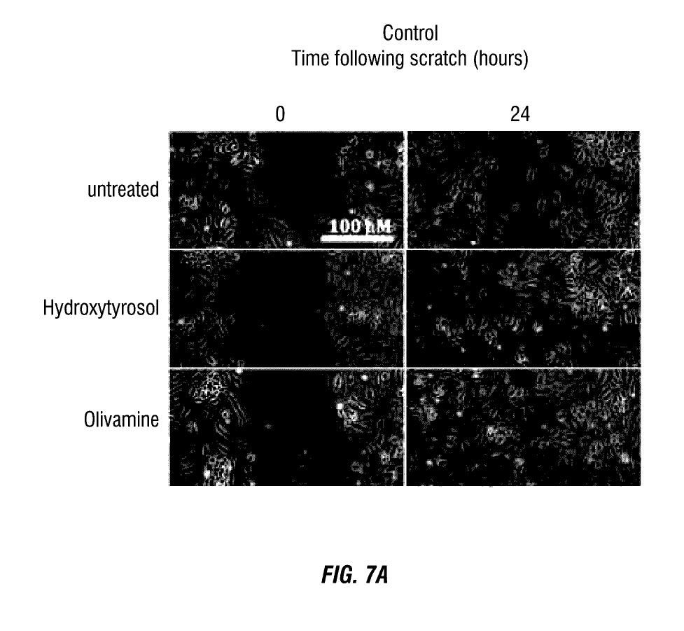

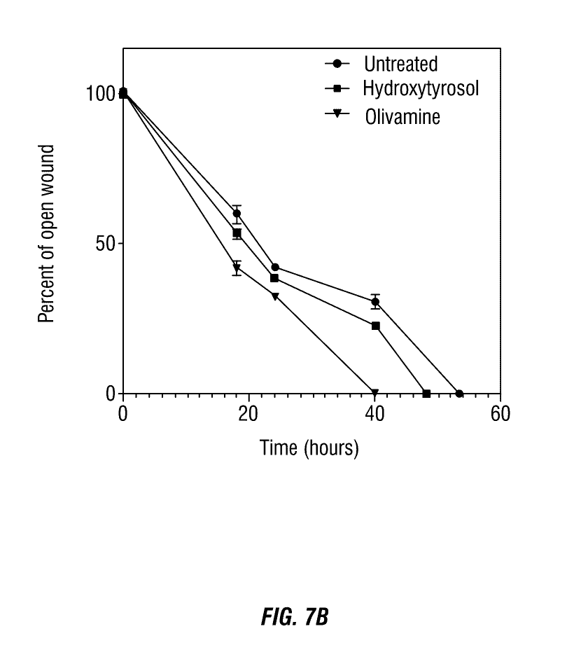

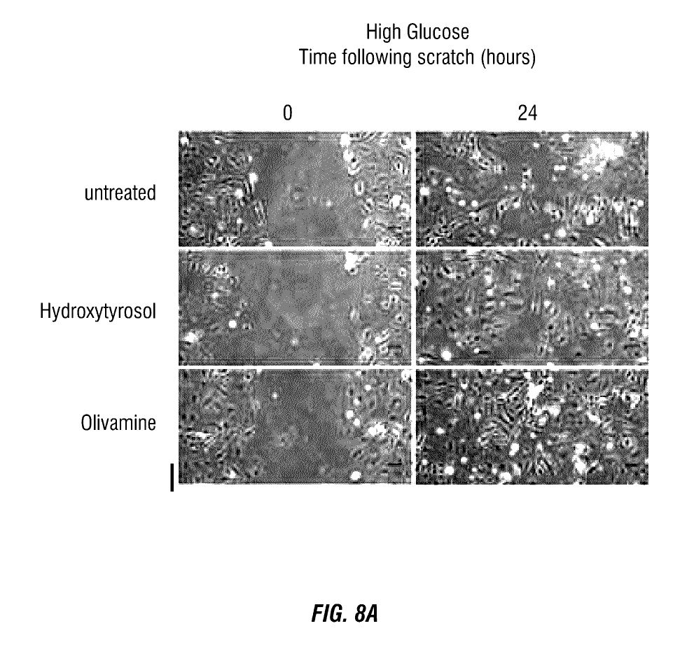

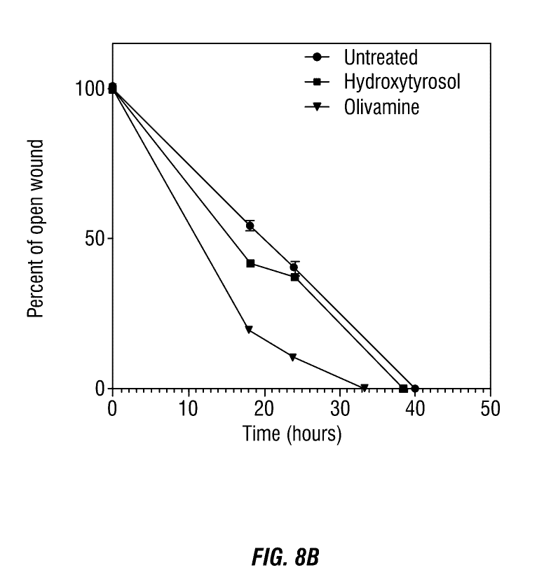

Images were taken at intervals and the wound gap was quantified over time using IMAGE® analysis software. For example, as shown in FIG. 6A, an initial image was taken at the time of injury (i.e. scratch) and the distance measured (as shown 734 microns); thereafter images are taken over incremental time periods (as shown in FIG. 6B the distance measured at 24 hours was 309 microns). The distances measured are depicted in the Figures by the horizontal solid lines indicating the distance between the gaps of the wound. 100 measurements were taken per image and a mean gap distance was determined. As shown in FIGS. 7-8 the initial wound and 24 hour measurement are shown by the images, under both control (FIG. 7A) and high glucose (FIG. 8A). In addition the reduction in percentage of open wound is further shown graphically for the control (FIG. 7B) and high glucose (FIG. 8B) conditions. The time for 50% of the wound to heal was quantified as well as the percentage of improvement compared to untreated cells.

The time for 50% of the wound to heal for the various treated tissues is shown in Table 2.

TABLE 2

| | Time for 50% wound | |
|---|---|---|
| Treatment | to heal (hours) | SEM |
| Untreated | 21.9 | 3.0 |
| Hydroxytyrosol | 19.7 | 2.1 |
| OLIVAMINE ® composition | 15.3 | 3.2 |

High Glucose

| Untreated | 21.0 | 2.1 |
|---|---|---|
| Hydroxytyrosol | 15.2 | 2.0 |
| OLIVAMINE ® composition | 8.6 | 1.0 |
| | Healing Improvement | |
| Treatment | (%) | SEM |
| Hydroxytyrosol | 10 | 2.1 |
| OLIVAMINE ® composition | 30 | 2.7 |

High Glucose

| | | |
|---|---|---|
| Hydroxytyrosol | 30 | 2.0 |
| OLIVAMINE ® composition | 60 | 0.9 |

The results indicate that OLIVAMINE® (compositions of hydroxytyrosol and oleuropein) composition improves cell migration by at least 60% in human umbilical vein endothelial cells following treatment with high glucose (i.e. toxic environments). This represents a significant improvement over the wound healing under the control/untreated (i.e. non-toxic environments) wherein a 30% improvement was observed. Beneficially, the use of OLIVAMINE® (compositions of hydroxytyrosol and oleuropein) composition under the high glucose conditions resulted in a decrease in healing time (time for 50% wound healing) from 15.3 hours to 8.6 hours. These results are still more significant in comparing the untreated HUVEC cells which had a 50% wound healing at 21.9 hours (and still 21 hours under the high glucose conditions).

According to the invention, the use of OLIVAMINE® (compositions of hydroxytyrosol and oleuropein) compositions (20 μM hydroxytyrosol, 80 μM oleuropein, 2 mM N-acetylcysteine, 50 μM L-proline, 2 mM glycine and 100 μM taurine) provide a significant improvement in wound healing using HUVEC cells, and provide an even further improvement in toxic wound environments.

### Example 3

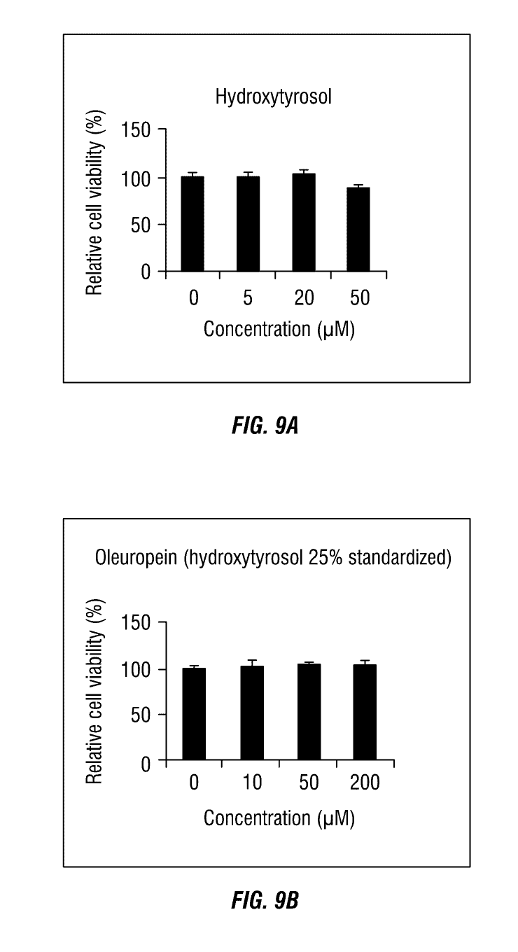

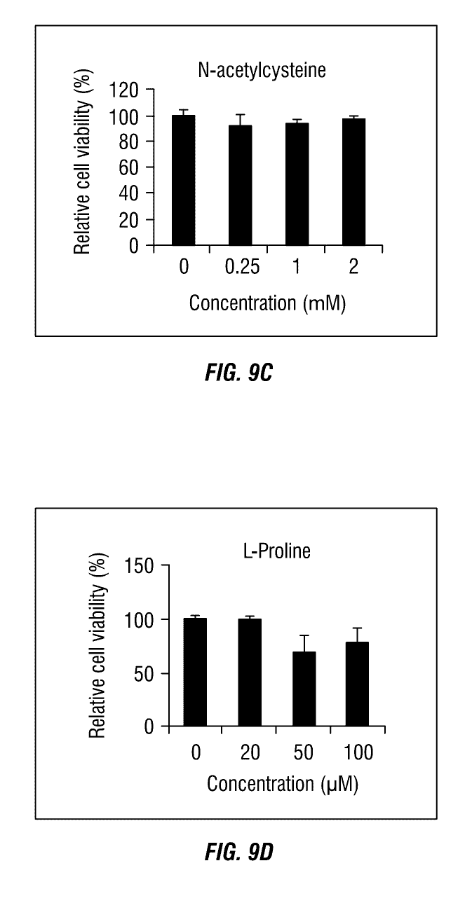

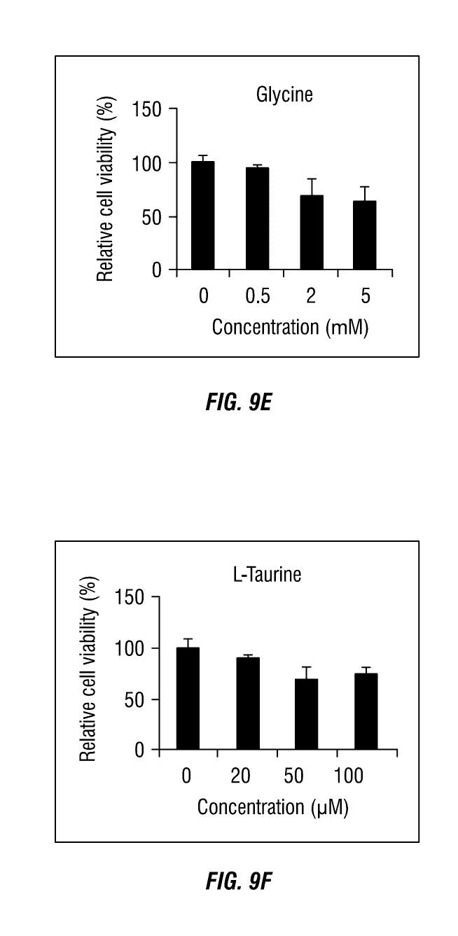

The precise ranges of each of the components utilized within the hydroxytyrosol and oleuropein-containing compositions were evaluated (six ingredients in relation to one another). FIGS. 9A-F illustrate the tested effects of the individual components of the claimed compositions on the growth of a vascularized endothelial cell line (e.g. cells of the stomach lining). Scientific analysis was carried out using a cell culture assay to determine relative cell viability for each ingredient. Four different concentrations were selected for initial concentrations (see Table 3) with a negative control. Individually the ingredients showed little (see 9B and 9C) to moderate (see 9A, 9D, 9E, 9F) impact on cellular growth.

TABLE 3
Initial Concentrations Tested

| Compound | | | | |
|---|---|---|---|---|
| Hydroxytyrosol (μM) | 0 | 5 | 20 | 50 |
| Oleuropein (hydroxytyrosol 25% | 0 | 10 | 50 | 200 |
| standardized) (μM) | | | | |
| N-acetyl-cysteine (mM) | 0 | 0.25 | 1 | 2 |
| L-Proline (μM) | 0 | 20 | 50 | 100 |
| Glycine (mM) | 0 | 0.5 | 2 | 5 |
| L-Taurine (μM) | 0 | 50 | 100 | 200 |

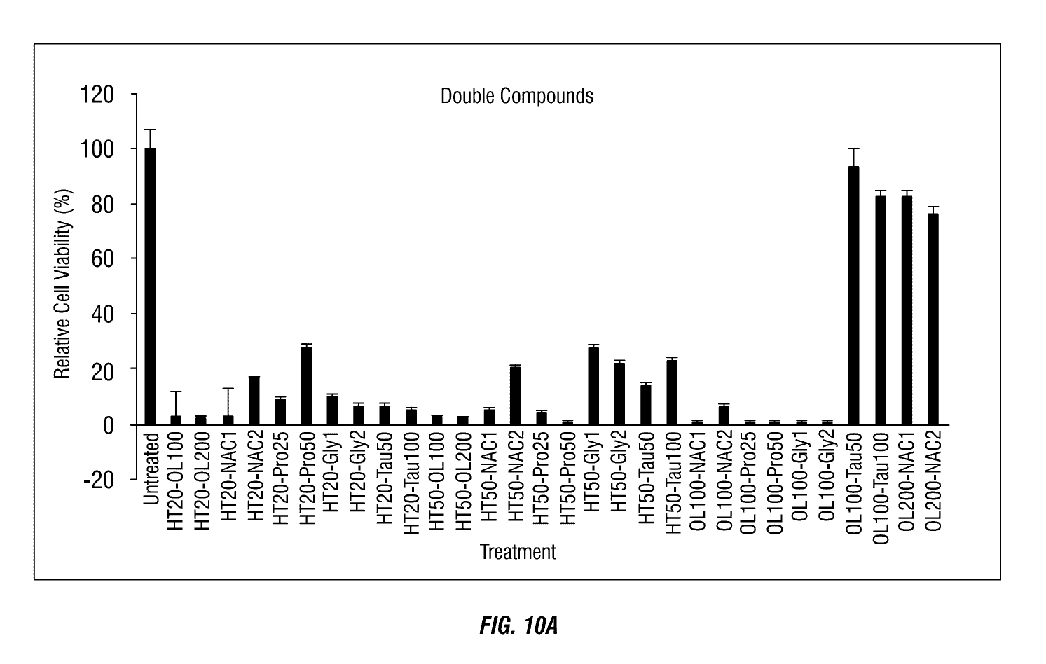

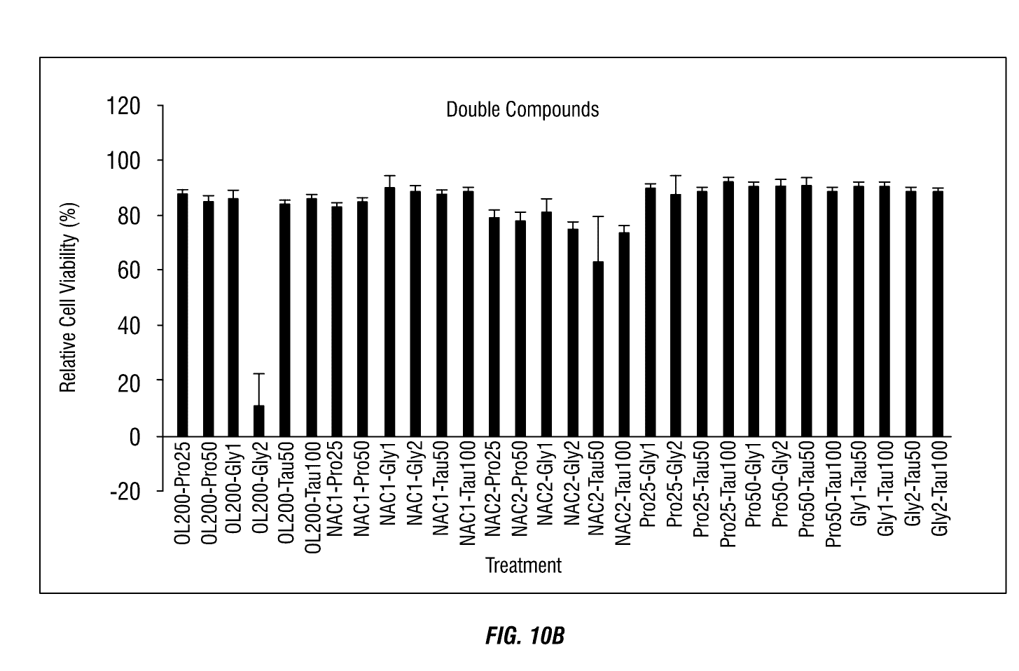

To calculate the needed ratios for the claimed compositions, a combination dosage study was conducted using the same testing method as described above on every two-ingredient combinations for the six ingredients (see FIG. 10). Ingredient concentrations were determined using the top two preforming concentrations from the initial study (shown in FIGS. 9A-F) with adjustments for oleuropein (hydroxytyrosol 25% standardized), glycine and L-Proline (see Table 4). The second combination and dosage study determined ingredient interactions and dosages for the final concentrations to be used for remaining test sets (see Table 5).

TABLE 4
Concentrations for Second Test Set

| Compounds | | |
|---|---|---|
| Hydroxytyrosol (μM) | 20 | 50 |
| Oleuropein (hydroxytyrosol 25% | 100 | 200 |
| standardized) (μM) | | |
| N-acetyl-cysteine (mM) | 1 | 2 |
| L-Proline (μM) | 25 | 50 |
| Glycine (mM) | 1 | 2 |
| L-Taurine (μM) | 50 | 100 |

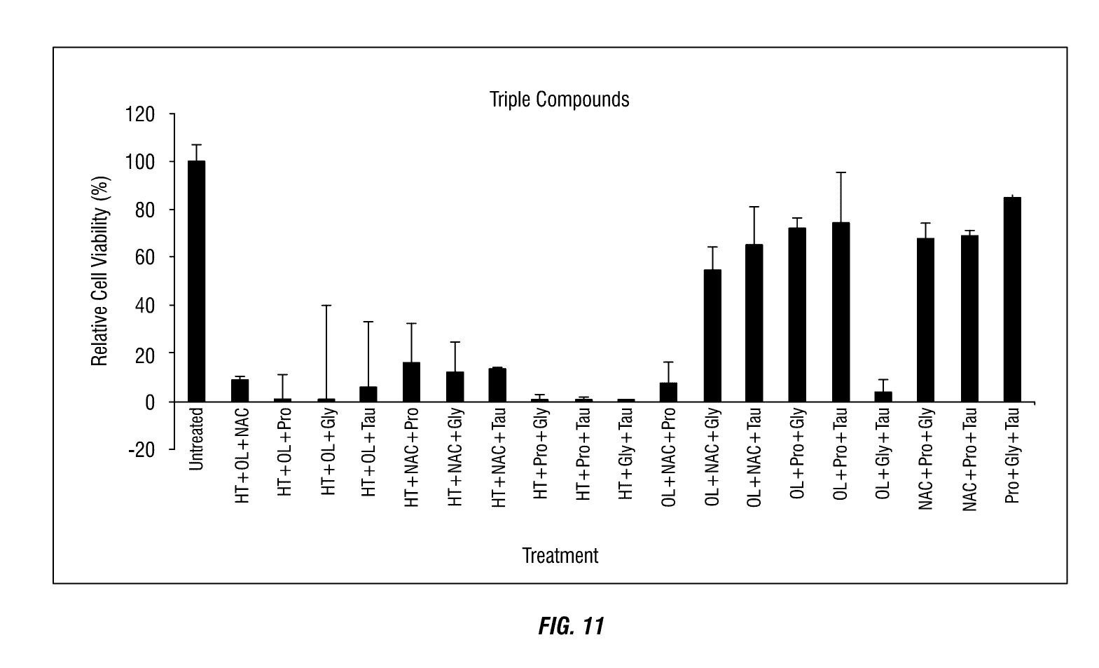

Further component amount/ratio evaluation was continued by using every unique triple ingredient combination for the six ingredients. Concentrations used were determined by the second test set (see Table 5). Ingredient synergy was observed for some combinations and no negative ingredient interactions for synergy were observed (see FIG. 11). The importance of hydroxytyrosol to the present art was determined.

TABLE 5
Concentrations for Third Test Set

| Triple | |
|---|---|
| Hydroxytyrosol (μM) | 50 |
| Oleuropein (hydroxytyrosol 25% standardized) | 200 |
| (μM) | |
| N-acetyl-cysteine (mM) | 2 |
| L-Proline (μM) | 50 |
| Glycine (mM) | 2 |
| L-Taurine (μM) | 100 |

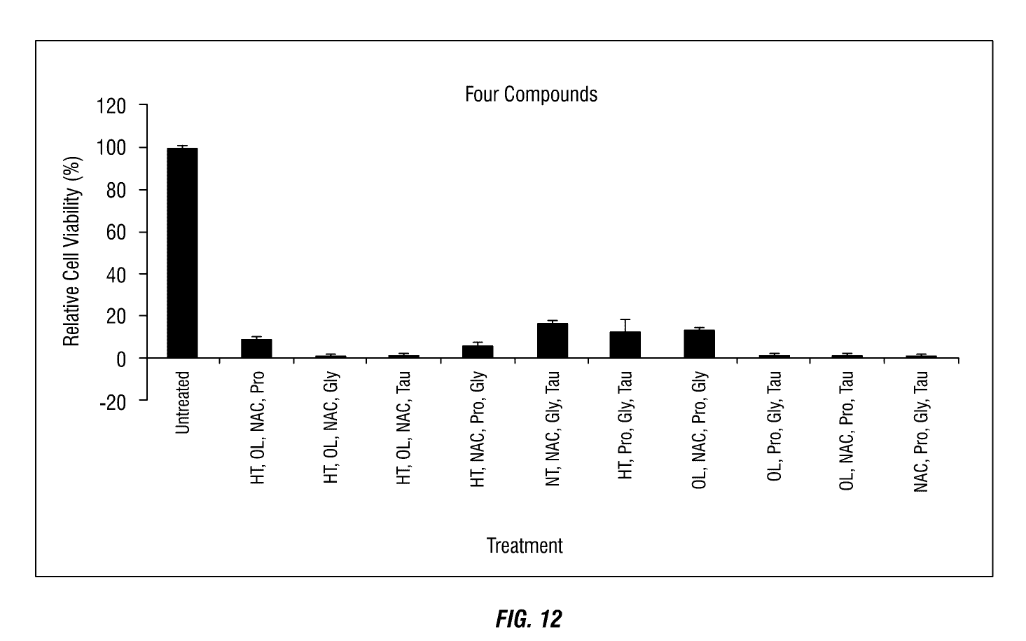

The four compound combinations were further performed following the same procedure of the previous test sets, with ingredient synergy becoming apparent and no negative ingredient interactions observed (see Table 6). The importance of L-proline to the present art was determined (see FIG. 12).

TABLE 6
Combinations and Concentrations of Four Compound Combination Test

| | | | | |
|---|---|---|---|---|
| Four Compounds | | | | |
| HT + Oleuropein + N-acetyl-cysteine + Proline | 50 (μM) | 200 (μM) | 2 (mM) | 50 (μM) |
| HT + Oleuropein + N-acetyl-cysteine + Glycine | 50 (μM) | 200 (μM) | 2 (mM) | 2 (mM) |
| HT + Oleuropein + N-acetyl-cysteine + Taurine | 50 (μM) | 200 (μM) | 2 (mM) | 100 (μM) |
| HT + N-acetyl-cysteine + Proline + Taurine | 50 (μM) | 2 (mM) | 50 (μM) | 100 (μM) |
| HT + Proline + Glycine + Taurine | 50 (μM) | 50 (μM) | 2 (mM) | 100 (μM) |
| Oleuropein + N-acetyl-cysteine + Proline + Glycine | 200 (μM) | 2 (mM) | 50 (μM) | 2 (mM) |
| Oleuropein + Proline + Glycine + Taurine | 200 (μM) | 50 (μM) | 2 (mM) | 100 (μM) |
| HT + N-acetyl-cysteine + Glycine + Taurine | 50 (μM) | 2 (mM) | 2 (mM) | 100 (μM) |
| Oleuropein + N-acetyl-cysteine + Proline + Taurine | 200 (μM) | 2 (mM) | 50 (μM) | 100 (μM) |
| N-acetyl-cysteine + Proline + Glycine + Taurine | 2 (mM) | 50 (μM) | 2 (mM) | 100 (μM) |

A fifth set of experiments following the previously described test procedure was used to determine the importance of six ingredients for the claimed compositions, excluding hydroxytyrosol and L-proline (established above) (see Table 7). The importance of the oleuropein (hydroxytyrosol 25% standardized)-hydroxytyrosol ingredient interaction to the synergy of the present art was observed (see FIG. 12).

TABLE 7
Combinations and Concentrations of Five Compound Combination Test

| | | | | | |
|---|---|---|---|---|---|
| Five Compounds | | | | | |
| HT + Oleuropein + N-acetyl-cysteine + | 50 (μM) | 200 (μM) | 2 (mM) | 50 (μM) | 2 (mM) |
| L-Proline + Glycine | | | | | |
| HT + Oleuropein + N-acetyl-cysteine + | 50 (μM) | 200 (μM) | 2 (mM) | 50 (μM) | 100 (μM) |
| L-Proline + L-Taurine | | | | | |
| HT + N-acetyl-cysteine + L-Proline + | 50 (μM) | 2 (mM) | 50 (μM) | 2 (mM) | 100 (μM) |
| Glycine + L-Taurine | | | | | |
| HT + Oleuropein + L-Proline + | 50 (μM) | 200 (μM) | 50 (μM) | 2 (mM) | 100 (μM) |
| Glycine + L-Taurine | | | | | |

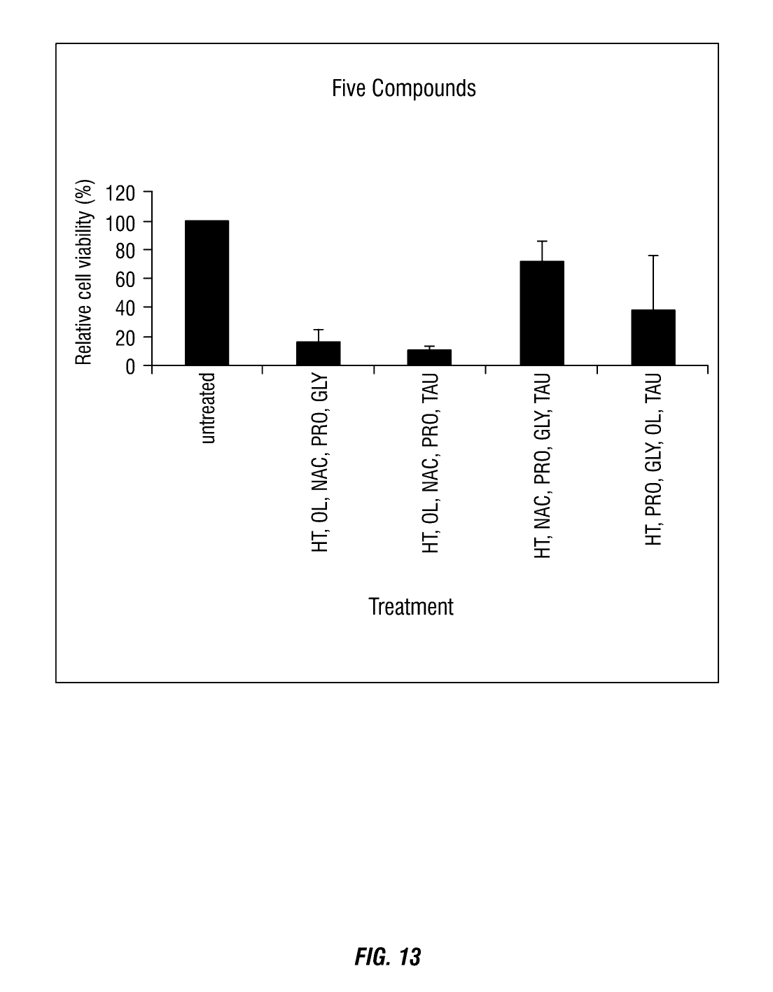

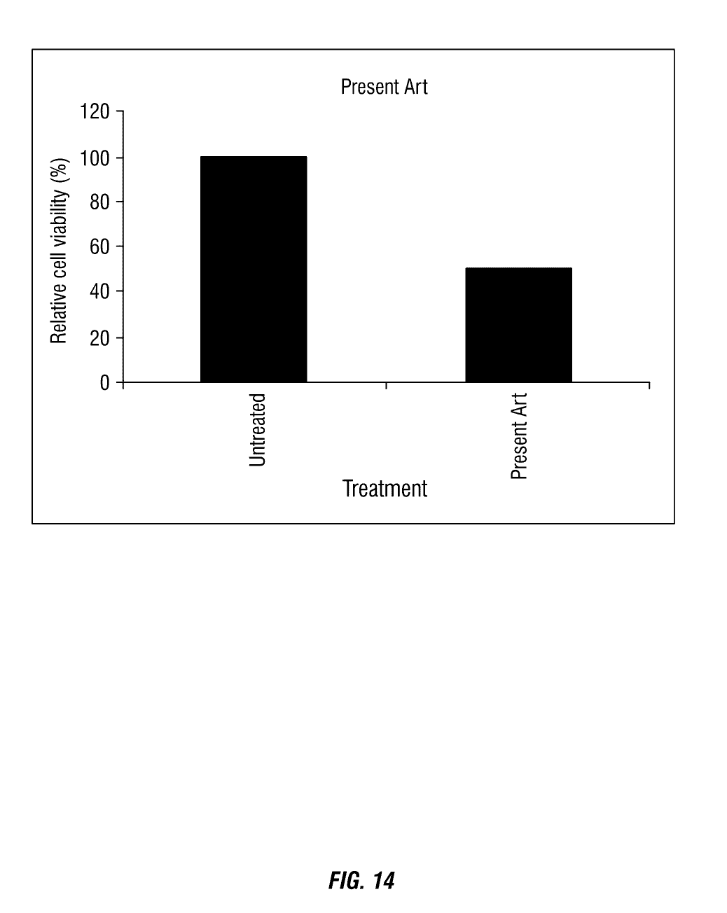

Three compound compositions were generated with the previously outlined component ingredient synergy (see Table 8). Radiation protection studies and relative cell viability in vitro testing was used to establish the final claimed compositions with established synergy in its impact of the cell growth of vascularized endothelial cells (see FIG. 13).

TABLE 8
Optimized Formulation 1 for Testing

| | Formulation 1 | | Formulation 2 | | Formulation 3 | |
|---|---|---|---|---|---|---|
| | Concentration/ | | Concentration/ | | Concentration/ | |
| | Percentage | | Percentage | | Percentage | |
| Compound | (μM)/(%) | | (μM)/(%) | | (μM)/(%) | |
| Hydroxytyrosol | 200 | (1.26) | 100 | (0.7) | 200 | (0.9) |
| Oleuropein | 800 | (17.7) | 400 | (9.78) | 800 | (12.66) |
| (hydroxytyrosol, 25% | | | | | | |
| standardized) | | | | | | |
| N-acetyl-cysteine | 8000 | (53.45) | 8000 | (59.05) | 8000 | (38.21) |
| L-Proline | 200 | (0.94) | 200 | (1.04) | 400 | (1.35) |
| Glycine | 8000 | (24.59) | 8000 | (27.16) | 20000 | (43.93) |
| L-Taurine | 400 | (2.05) | 400 | (2.26) | 800 | (2.93) |

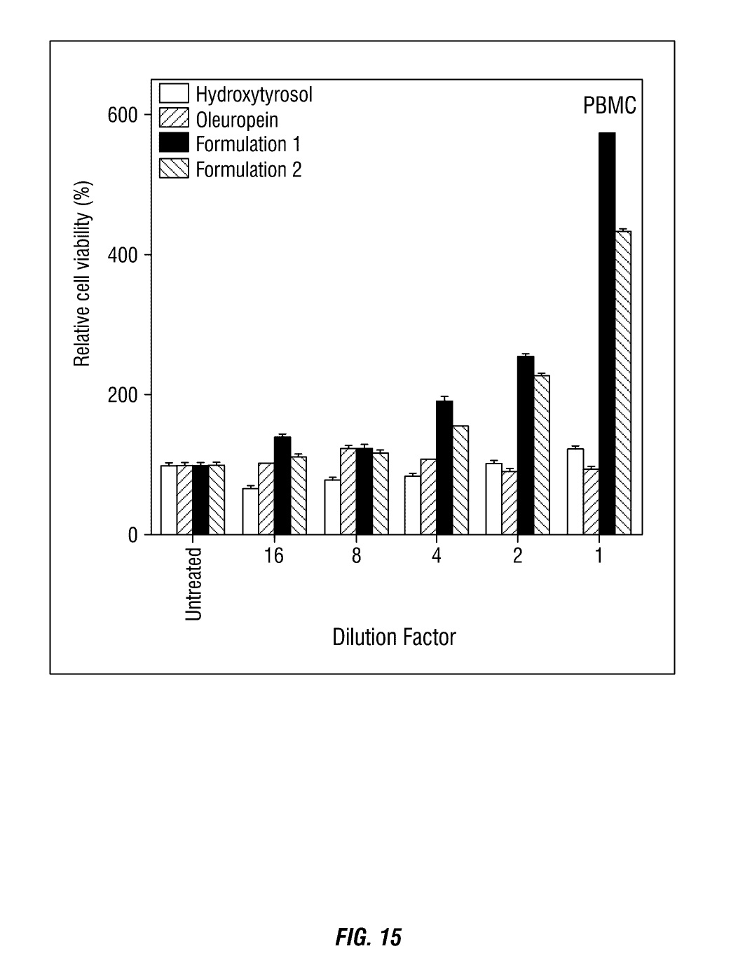

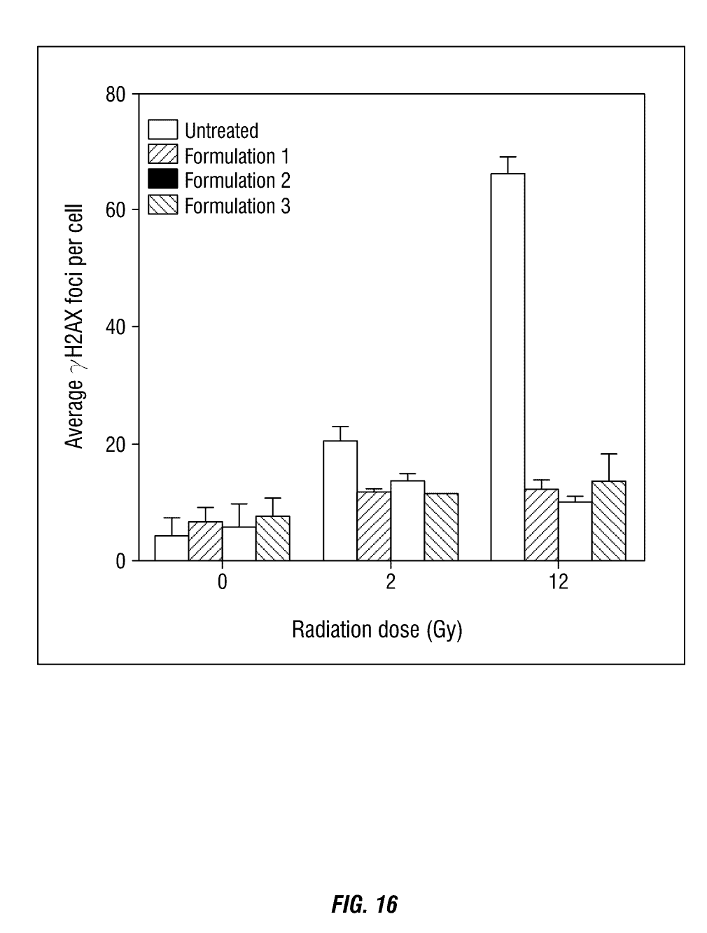

The three formulations were analyzed for compound synergy and cell proliferative properties of healthy cells. The first test set was a dilution test conducted on compounds 1 and 2, and the individual ingredients hydroxytyrosol and oleuropein (hydroxytyrosol 25% standardized) and their impact on relative cell viability. Compound 1 showed a synergistic ability to promote the growth of healthy cells (see FIG. 15). The second test set conducted was a radiation protection study of healthy cells conducted on Formulations 1, 2, and 3 and the individual ingredients hydroxytyrosol and oleuropein (hydroxytyrosol 25% standardized). The average γH2AX foci per cell was used as a measure of radiation induced DNA damage in the cell. Formulation 1 showed the best ability to protect cells in doses of 2 Gy and showed moderately less protective effect in doses of 12 Gy as compared to Compound 2 (see FIG. 16).

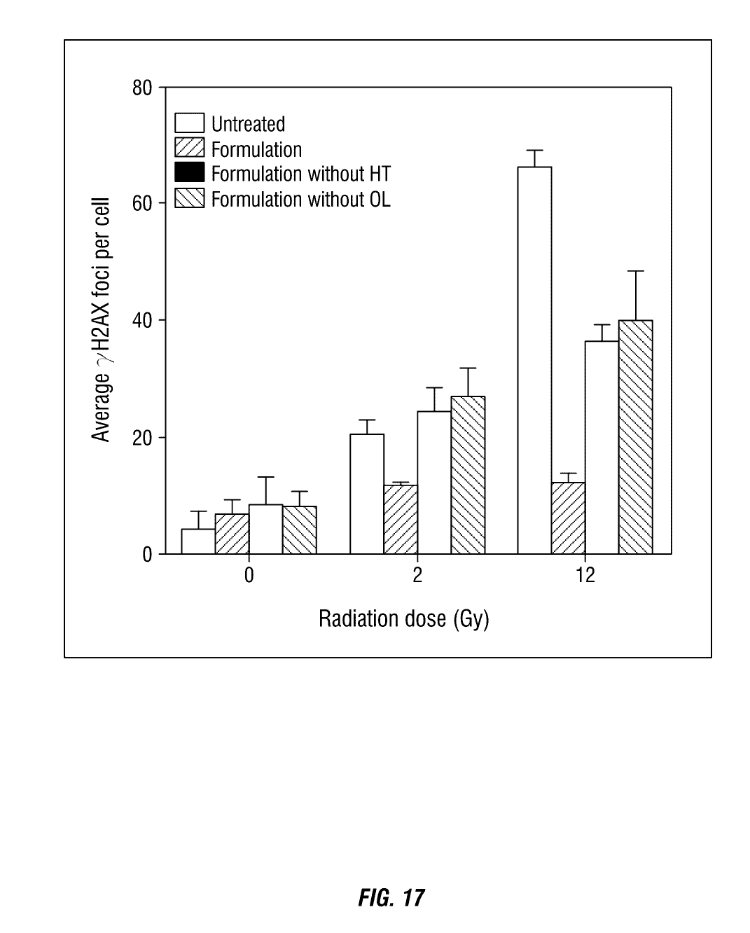

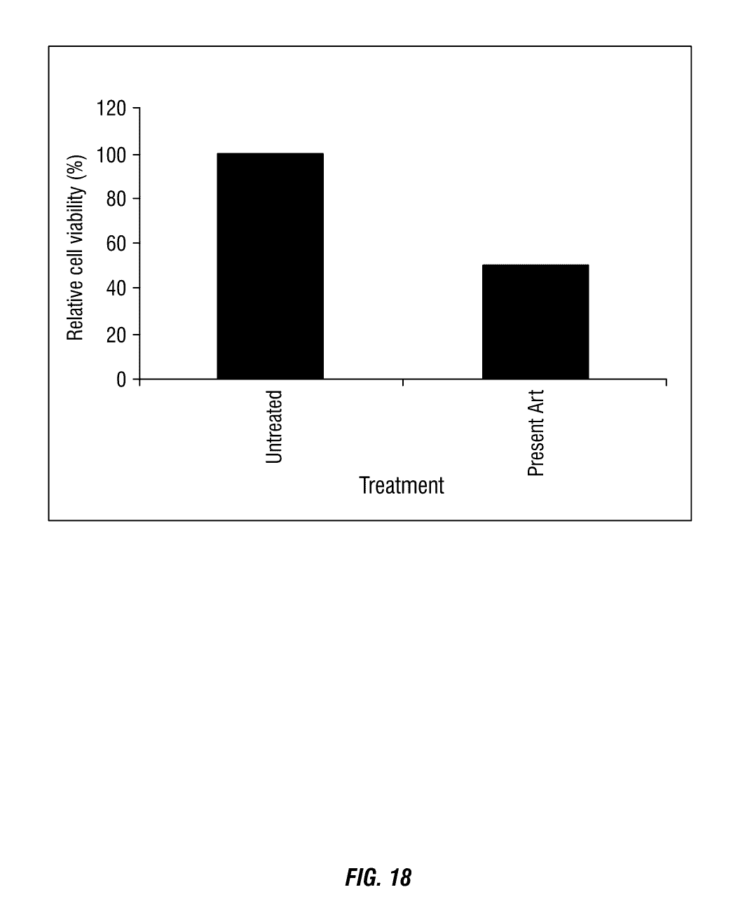

Based on its synergistic abilities to promote healthy cell growth and to protect healthy cells from DNA damage when exposed to radiation Formulation 1 demonstrates the greatest cytoprotective effect for healthy cells (see Table 9). Compound synergy was determined using the same radiation protection study as described above, conducted on two formulation without the ingredients hydroxytyrosol and oleuropein (hydroxytyrosol 25% standardized) respectively in comparison to Formulation 1. Formulation 1 showed synergistic radiation protective effects as compared to the formulations without hydroxytyrosol and oleuropein (hydroxytyrosol 25% standardized) (see FIG. 17). Synergy was further confirmed with a relative cell viability assay using the same procedure as described for the previous tests. The formulation showed significant synergistic impact on the relative cell viability of vascularized endothelial cells (see FIG. 18).

TABLE 9

| | Time for 50% wound | |
|---|---|---|
| Treatment | to heal (hours) | SD |
| Untreated | 26.01 | 0.71 |
| Present Art | 17.75 | 1.06 |
| Hydroxytyrosol | 19.25 | 1.84 |
| Oleuropein | 25.62 | 2.17 |
| N-acetylcysteine | 28.87 | 1.95 |
| L-proline | 23.24 | 1.53 |
| Glycine | 20.73 | 2.08 |
| L-Taurine | 22.5 | 1.83 |

Present Art Without . . .

| | | |
|---|---|---|
| Hydroxytyrosol | 26.59 | 4.29 |
| Oleuropein | 31.25 | 3.66 |
| N-acetylcysteine | 56.99 | 6.68 |
| L-proline | 19.84 | 0.51 |
| Glycine | 28.58 | 0.66 |
| L-Taurine | 30.28 | 1.81 |

The various outlined demonstrations of the criticality of the claimed amounts/ratios of the compositions according to the present invention show the unexpected results obtained from the strong synergistic effect obtained by the combinations within the formulation.

### Example 4

### Methods

### Cell Culture

Human microvascular endothelial cells (HMEC-1) transfected with the rous sarcoma virus were obtained from the American Type Culture Collection (Manassas, Va., USA) and grown as monolayers in MCDB-131 medium supplemented with L-glutamine, epidermal growth factor (EGF), heparin, hydrocortisone and Gluta-MAX. Cells were maintained in the exponential growth phase in T75 cm² vented culture flasks and passaged by 0.05% (v/v) trypsin-EDTA before seeding; at 37° C., 5% (v/v) CO₂.

Human umbilical vein endothelial cells (HUVECs) were cultured in endothelial cell growth medium (ECM) supplemented with 100 U/ml penicillin, 100 μg/ml streptomycin, 0.25 μg/ml fungizone, 2 mM L-glutamine, 5 U/ml heparin, 30-50 μg/ml endothelial cell growth supplement. All cells were passaged by typsination and seeded into 6 well culture dishes at cell densities of 1×10⁶ cells per well; at 37° C., 5% (v/v) CO₂.

Human peripheral blood mononuclear cells (PBMCs) were fractionated using the Ficoll Plaque fractionation method from whole blood. Cells were harvested fresh on the day of the experiments and maintained in complete-RPMI-1640 medium supplemented with 10% FBS, 2 mM L-glutamine and 20 μg/mL gentamicin at 37° C., 5% (v/v) CO₂. To stimulate the PBMC cell cycle, cells were treated with 25 ng/mL phorbol myristate acetate (PMA) for 4 hours prior to treatment with OLIVAMINE® (compositions of hydroxytyrosol and oleuropein) composition.

Angiogenesis (Vascular Tube Formation) Assay

Cells were seeded at 1×10⁵ cells per well in a 48 well, flat bottom culture plate and allowed to attached overnight. Cells were treated with Formulation 1 (dilution factor=4, Table 8) for 24 hours prior to typsination by 0.05% trypsin EDTA. The cells were seeded at 10,000 cells per well in the ibidi μ-treat angiogenesis culture slide coated with 0.8% agarose culture medium mix according to the manufacturer's protocols. Cells were imaged immediately (0 hours) and at 6 hours, using a light microscope using a 10× objective. Tube length was analyzed in Image J.

Cell Viability

A crystal violet cell viability assay was performed to determine the cytotoxic effects of OLIVAMINE® (compositions of hydroxytyrosol and oleuropein) composition on HMEC-1 cells. Briefly, cells were seeded on a 24-well plate and treated for 24 h dose-titration concentrations of HT (0-200 μM), OL (0-800 μM), OLIVAMINE® (compositions of hydroxytyrosol and oleuropein) composition (McCord formulation 1, dilution factor=4×, Table 8). Following treatment, cells were washed twice with 0.9% saline and fixed with 2004 of 4% neutral formalin for 15-30 min. Cells were then stained with 2004 of 0.01% (w/v) crystal violet for 30 min, washed with dH2O and left to dry under a fume hood overnight. Cells were then lysed with 4004/well of lysis buffer for 15 min on a rotating platform and the absorbance of each well was measured at the 560 nm wavelength using a microplate spectrophotometer.

Mitochondrial Stress Test Assay

HMEC-1 cells and circulating white blood cells PBMCs were seeded at 7.5×10⁵ cells per well in a XF96 cell culture microplate and treated with Formulation 1 (dilution factor=4, Table 8) for 24 hours. Oxygen consumption rate (OCR) was determined using an XFe96 Extracellular Flux Analyzer. Briefly, the cell culture media was removed from the XF96 cell culture microplate and replaced with 1804 of XF Assay Medium, before being incubated at 37° C., with no CO₂, for 1 h prior to assay run. The four stress test compounds; Oligomycin, carbonyl cyanide-4-(trifluoromethoxy) phenylhydrazone (FCCP), Antimycin A and Rotenone were freshly prepared to 1 μM in Seahorse assay medium and 254 of each stress test compound was added to the hydrated 96-well cartridge plate approximately 1h before the assay run. The OCR was measured at basal conditions, and after each subsequent injection of the four stress test compounds. A bradford protein assay was performed immediately after the Seahorse assay to normalise the data according to cell mass (μg/μl).

### Results

OLIVAMINE® (Compositions of Hydroxytyrosol and Oleuropein) Composition Improves Tube Formation of Vascular Endothelial Cells

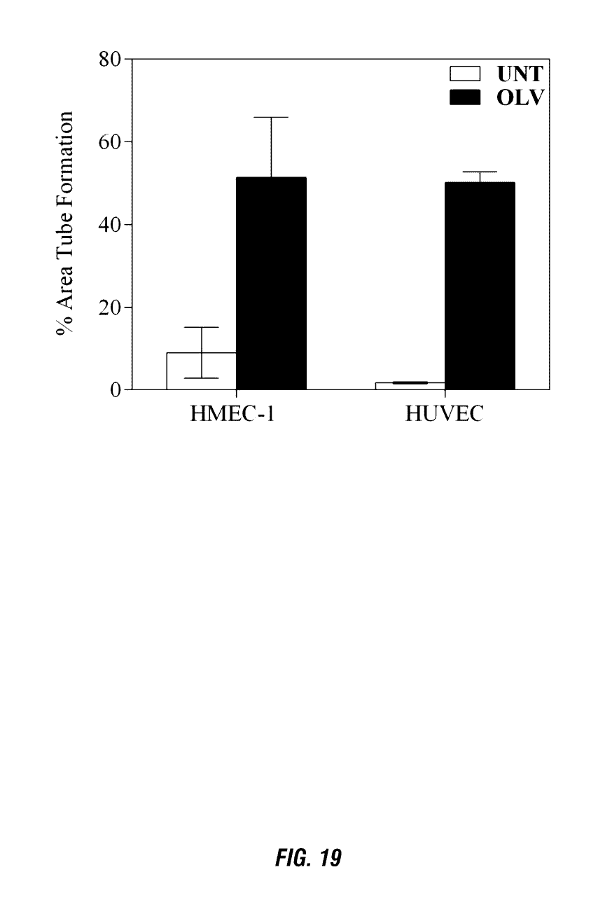

Vascular tube formation assays was performed to assess the vasculogenic activity of HT and/or OL-containing formulations in HMEC-1 and HUVEC vascular endothelial cells. Cells were treated for 24 hours with Formulation 1 (dilution factor 4, Table 8) and transferred to an agarose medium in order to sufficiently assess vascular tube formation using the μ-slide ibidi angiogenesis assay and monitored over a 6 hour period. The results demonstrate OLIVAMINE® (compositions of hydroxytyrosol and oleuropein) composition enhanced neovascularisation when compared to untreated cells (FIG. 19). The findings indicate that OLIVAMINE® (compositions of hydroxytyrosol and oleuropein) composition has a remarkable effect on tube formation in vascular HMEC-1 and HUVEC cells.

Effect of Olive Phenols on Cell Viability in Vascular Endothelial Cells

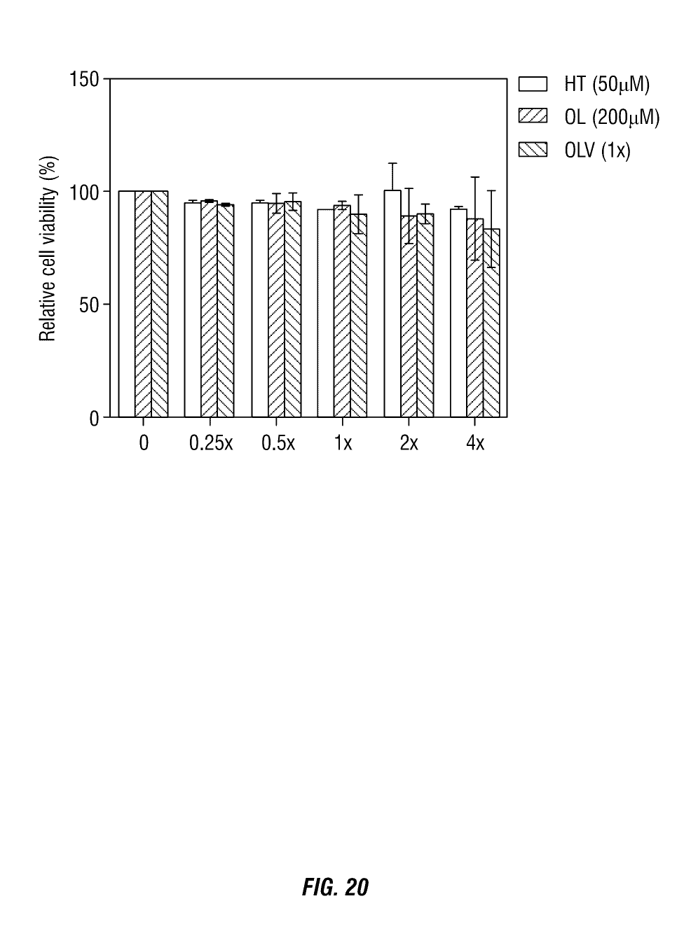

To determine whether olive phenols influence cell proliferation, a crystal violet cell viability analysis was conducted. The relative cell viability of HMEC-1 cells was investigated. Cells were incubated with varying concentrations of HT (0-200 μM), OL (0-800 μM) and Formulation 1 (Table 8, 0-4×) 24 hours. The findings indicated that HT, OL and Formulation 1 have no significantly effects on cell proliferation at these concentration range (FIG. 20). Hence it was conferred that no cytotoxic effects were induced.

OLIVAMINE® (Compositions of Hydroxytyrosol and Oleuropein) Composition Enhances Mitochondrial Function as Indicated by Increased Oxygen Consumption Rates.

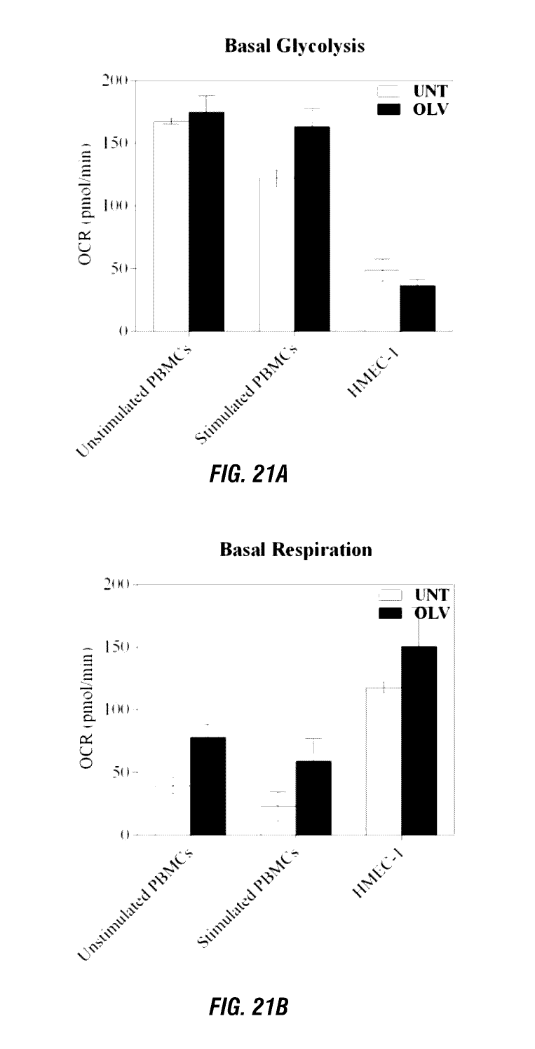

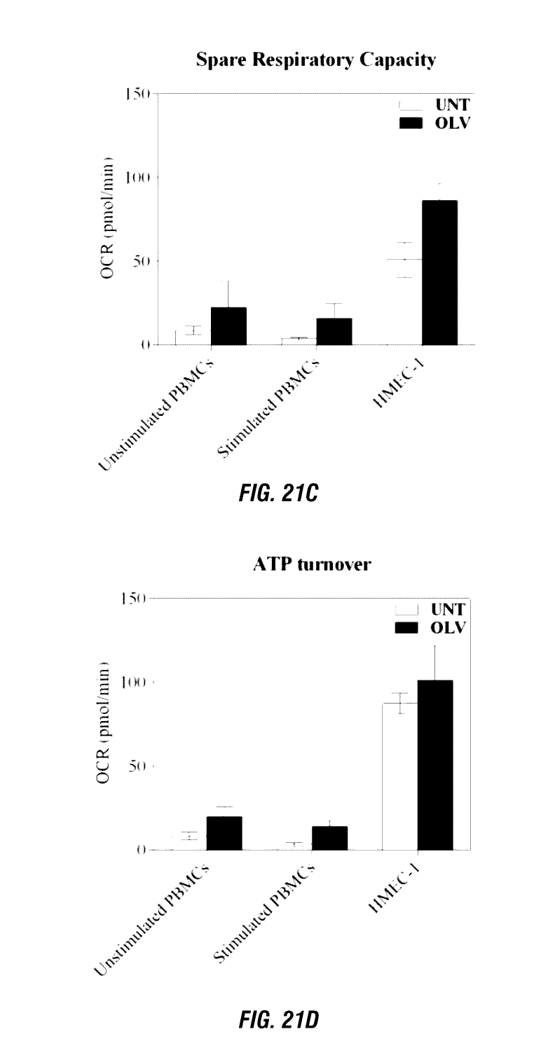

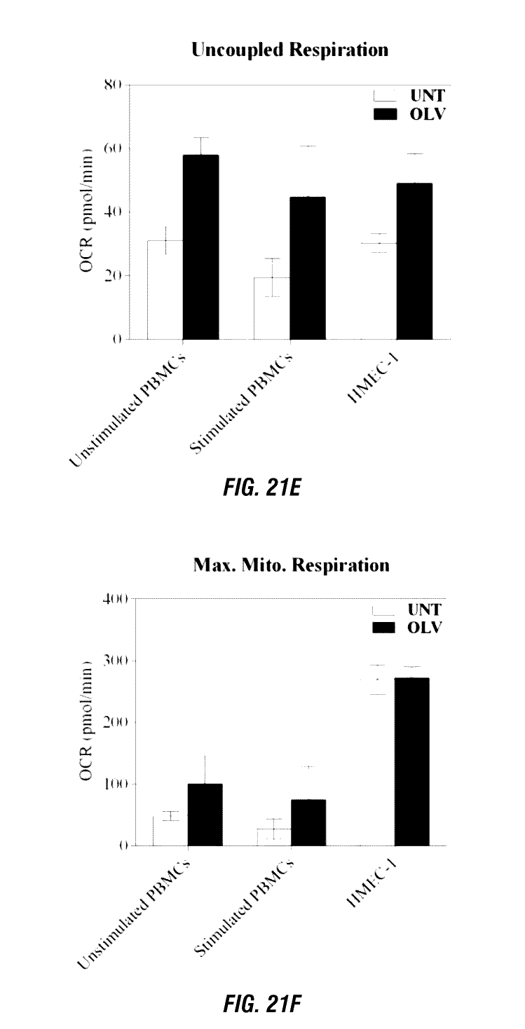

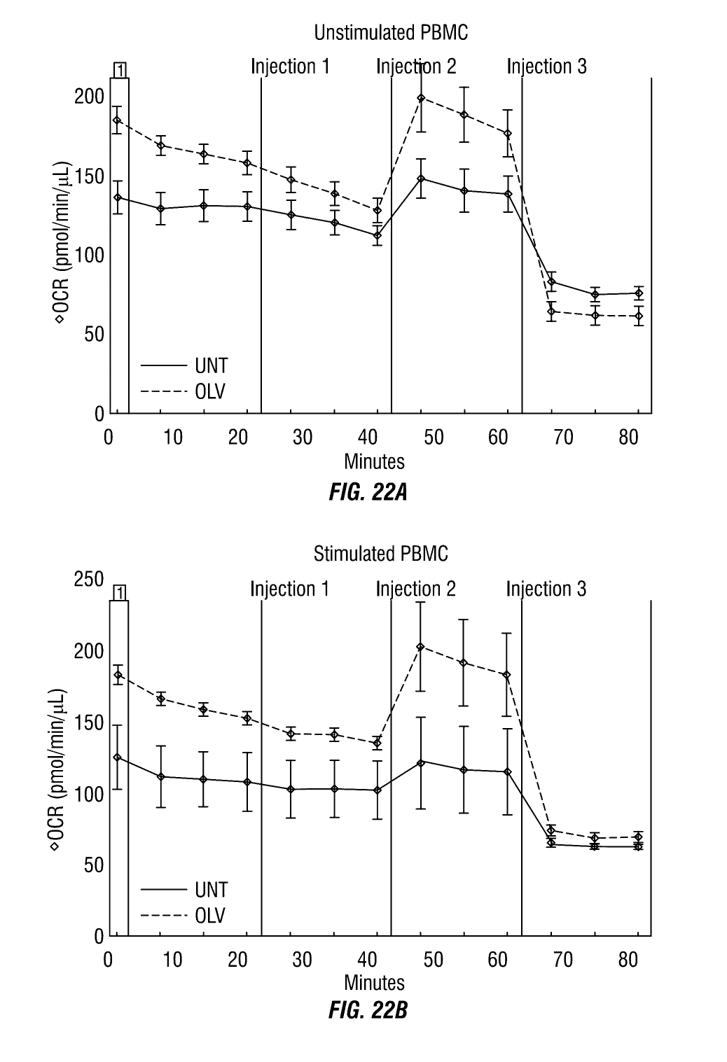

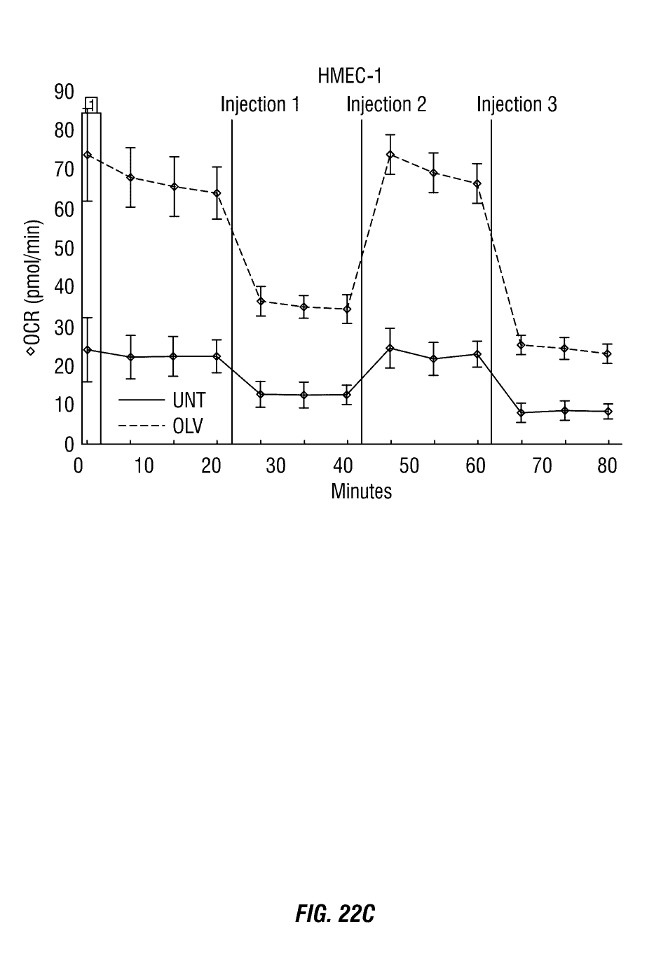

The bioenergetic actions of olive phenols in vascular HMEC-1 cells and circulating mononuclear cells (PBMCs) was investigated using a SEAHORSE EXTRACELLULAR FLUX ANALYSER® system which measured the oxygen consumption rate (OCR) (indicative of mitochondrial respiration) and the extracellular acidification rate (ECAR) (reflective of glycolysis) in the intact cells. Cells were pre-treated with Formulation 1 (0-4×) for 24 hours prior to performing the assay and all data was normalized to protein content. Investigation of mitochondrial oxygen consumption and electron transport chain complex activities was determined by changes in the OCR under different respiratory conditions defined by the sequential injections of oligomycin (ATP-synthase inhibitor), FCCP (proton gradient uncoupler) and rotenone plus antimycin A (complex I inhibitor) in real time. From the differential responses to the complex inhibitors and uncoupler, the basal glycolysis, basal respiration, uncoupled respiration, spare respiratory capacity, ATP production and maximal mitochondrial respiration where measured (FIGS. 21 and 22).

Basal glycolysis and the extracellular pH were measured, which conferred proton production levels to glycolysis. The glycolytic rate of stimulated PBMCs increased following treatment with Formulation 1 but remained unchanged in the immortalised HMEC-1 cells and unstimulated PBMC cells suggesting Formulation 1 has little effect on glycolysis.

The basal respiration reflecting oxygen consumption used to meet cellular ATP demand increased following treatment with Formulation 1 in all cell lines. ATP production

(ATP turnover) which was calculated by the proportion of oxygen consumption used to drive mitochondrial ATP production also increased following Formulation 1 pre-treatment in all cell lines. Uncoupled respiration represents the remaining basal respiration not coupled to ATP production following an H⁺ proton leak within the electron transport chain. Pre-treatment with Formulation 1 increased uncoupled respiration in all cell lines indicating a possible non-mitochondrial respiration process allowing the cells to respire under mitochondrial stress.

Maximal respiration was induced by the FCCP injection, which permeabilizes the mitochondrial inner membrane, allowing protons to cross into the matrix and accelerate the action of the ETC. This measure is reflective of the cells bioenergetic capacity. Spare respiratory capacity provides information on how closely the cell is working to its maximal capacity during basal respiration and indicates the capability of the cell to respond to an energetic demand. We showed olive phenols augment maximal respiration and their spare respiratory capacity in HMEC-1 and PBMC cells.

The inventions being thus described, it will be obvious that the same may be varied in many ways. Such variations are not to be regarded as a departure from the spirit and scope of the inventions and all such modifications are intended to be included within the scope of the following claims.

1: A composition for accelerating wound closure to improve wound healing comprising:
an effective amount of hydroxytyrosol, or oleuropein, or a combination of hydroxytyrosol and oleuropein; and
an effective amount of primary vascular endothelial cells.

2: The composition of claim 1, wherein the administration of the composition reduces the time required for healing of the wound by at least about 30% in comparison to a wound treated with the endothelial cells alone.

3. (canceled)

4: The composition of claim 1, wherein the primary vascular endothelial cells are derived from umbilical cord blood and are umbilical cord stem cells, or wherein said endothelial cells are provided in the form of a medium conditioned by endothelial cells or growth factors which are beneficially increased as a result of an endothelial stem cell transplant.

5: The composition of claim 1 wherein said primary vascular endothelial cells are human endothelial cells.

6: The composition of claim 5 wherein said human endothelial cells are microvascular endothelial cells.

7: The composition according to claim 1, wherein the ratio of hydroxytyrosol to oleuropein is front about 1:1 to about 10:1.

8: The composition according to claim 1, wherein the composition further comprises N-acetyl cysteine, glycine, taurine and L-proline.

9: The composition according to claim 8, wherein the weight ratio of N-acetyl cysteine to hydroxytyrosol is between 1:1 and 50:1, wherein the weight ratio of glycine to hydroxytyrosol is between 1:1 and 50:1, wherein the ratio of taurine to hydroxytyrosol is between 1:1 and 50:1, and wherein the weight ratio of L-proline to hydroxytyrosol is between 1:1 and 20:1.

10: The composition according to claim 9, wherein the composition has a concentration of hydroxytyrosol less than 225 μM and a concentration of oleuropein less than 900 μM.

11: The composition according to claim 9, wherein the composition has a concentration of hydroxytyrosol less than 15 μM and a concentration of oleuropein less than 60 μM.

12: A method for improving wound healing comprising:
topically administering or transplanting to a subject in need of wound healing a composition comprising hydroxytyrosol and oleuropein and primary human vascular endothelial cells; and
reducing the time required for wound healing at least about 30% in comparison to a wound treated with the endothelial cells alone.

13: The method according to claim 12, wherein the wound healing further comprises promoting keratinocyte and fibroblast proliferation and/or neovascularization.

14: The method according to claim 12, wherein the endothelial cells are derived from umbilical cord blood and are umbilical cord stem cells, or a cultured medium conditioned by said umbilical cord stem cells.

15: The method according to claim 12, wherein the ratio of hydroxytyrosol to oleuropein is from about 1:1 to about 10:1.

16: The method according to claim 12, wherein the composition further comprises N-acetyl cysteine, glycine, taurine and L-proline.

17: The method according to claim 16, wherein the weight ratio of N-acetyl cysteine to hydroxytyrosol is between 1:1 and 50:1, wherein the weight ratio of glycine to hydroxytyrosol is between 1:1 and 50:1, wherein the ratio of taurine to hydroxytyrosol is between 1:1 and 50:1, and wherein the weight ratio of L-proline to hydroxytyrosol is between 1:1 and 20:1.

18: The method according to claim 12, wherein weight ratio N-acetyl cysteine to hydroxytyrosol is between 10:1 and 30:1, the weight ratio glycine to hydroxytyrosol is between 30:1 and 40:1, the weight ratio of taurine to hydroxytyrosol is between 20:1 and 50:1, and wherein the weight ratio of L-proline to hydroxytyrosol is between 1:1 and 10:1.

19: The method according to claim 12, wherein the time required for healing at least 50% of a wound is reduced to below at least 8 hours.

20: A composition for accelerating wound closure to improve wound healing comprising:
an effective amount of hydroxytyrosol, or oleuropein, or a combination of hydroxytyrosol and oleuropein; and
an effective amount of primary human vascular endothelial cells.
